(19) United States
(12) Patent Application Publication
(10) Pub. No.: US 2026/0279511 A1
(43) Pub. Date: Sep. 17, 2026

(54) PAN-SUBSTRUCTURAL HIERARCHICAL ALGORITHM FOR EXTENSIVE CHEMICAL SPACE ANALYSIS

(71) Applicants: Insilico Medicine IP Limited, Hong Kong (HK); Insilico Medicine AI Limited, Abu Dhabi (AE)

(72) Inventors: Bogdan Zagribelnyy, Abu Dhabi (AE); Sergei Fedorchenko, Zurich (CH); Nikita Bondarev, Abu Dhabi (AE); Aleksandrs Zavoronkovs, Hong Kong (HK)

(21) Appl. No.: 19/564,938

(22) Filed: Mar. 12, 2026

Related U.S. Application Data
(60) Provisional application No. 63/772,147, filed on Mar. 14, 2025.

(51) Int. Cl. G16C 20/64; G16C 20/62

(52) U.S. Cl. CPC G16C 20/64; G16C 20/62

(57) ABSTRACT

A method for improving synthetic accessibility analysis of molecular structures may be implemented. A hierarchical library of molecule substructures representing possible molecular fragments may be generated. Statistics on substructure occurrences in reference chemical datasets may be collected. Synthetically irrelevant substructures may be identified based on the occurrence statistics, wherein low occurrence statistics may indicate the synthetically irrelevant substructures. The library of irrelevant substructures may be clustered and optimized. A group of molecular structures may be filtered using a hierarchical algorithm to identify those containing irrelevant substructures. The molecular structures containing irrelevant substructures may be removed from the group of molecular structures. A filtered set of molecular structures may be obtained by removing molecular structures having synthetically irrelevant substructures.

### CROSS-REFERENCE TO RELATED APPLICATIONS

This patent application claims priority to U.S. Provisional Application No. 63/772,147 filed Mar. 14, 2025, which provisional is incorporated herein by specific reference in its entirety.

### BACKGROUND

### Field

The present disclosure relates generally to the field of computational chemistry and cheminformatics, particularly to computer-implemented methods and systems for analyzing chemical structures and predicting synthetic accessibility of molecular compounds. More specifically, the disclosure pertains to hierarchical data structures and filtering algorithms for evaluating molecular fragments within chemical space, with applications in computer-aided drug design and generative chemistry platforms.

### Description of Related Art

One of the central challenges in the discovery of novel molecular structures with potential biological activity is defining the subset of chemical space that is synthetically accessible. Early attempts to estimate this subspace were made by the cheminformatician D. Weininger, who approximated the number of synthetically feasible organic compounds with a molecular mass ≤1000 Da to be on the order of 10¹⁸⁰ [1]. In parallel, estimates of the total number of possible drug-like compounds span a wide range, from 10²³ to 10⁶⁰ molecular structures [2,3]. More recently, prior estimation approaches and the structural constraints applied therein were critically reassessed, leading to an updated estimate of approximately 10³³ molecules for the drug-like chemical space, defined as compounds containing no more than 36 atoms and having a molecular mass ≤500 Da [4]. Even at this reduced scale, it is evident that any form of exhaustive or even coarse-grained screening of such a chemical space remains computationally infeasible [5].

The rapid advancement of computer technologies in the 1990s enabled the emergence of the first automated approaches for de novo drug design, along with methods now broadly classified as generative chemistry. In its most general sense, generative chemistry encompasses computational techniques capable of automatically or semi-automatically producing organic molecular structures with predefined properties. Contemporary machine learning methods, combined with combinatorial in silico synthesis, make it possible to generate hundreds of thousands of candidate molecules. However, the proliferation of such approaches has brought renewed attention to a critical bottleneck: the evaluation of generated compounds, with particular emphasis on synthetic accessibility (SA), which remains the most universal prerequisite for practical applicability [5,6]. Existing strategies for modeling synthetic accessibility—primarily automated retrosynthetic analysis and data-driven scoring methods—are subject to well-recognized limitations, including high computational cost and limited interpretability [5].

The integration of deep learning techniques into drug discovery has led to an exponential increase in research focused on the development of new generative models [7,8]. Although substantial challenges remain [9], and further methodological advances are required to fully unlock the potential of generative modeling [10], automated de novo generation of molecular structures with tailored properties has already become an essential component of modern rational drug design workflows [6,11,12].

Nevertheless, it is widely acknowledged that the practical utility of even the most advanced generative models is often undermined by the unrealistic nature of the molecular structures they produce, despite achieving high scores on commonly used quantitative metrics such as novelty or compliance with drug-likeness filters [13]. In practice, synthetic accessibility frequently serves as the decisive criterion for prioritizing and selecting compounds for experimental synthesis [14], as the overarching objective of de novo molecular generation is to reduce cost and accelerate the identification of viable hit compounds. Consequently, low synthesis cost and rapid compound production are critically important attributes, underscoring the need for robust and reliable methods to model and predict synthetic accessibility.

### SUMMARY

In some embodiments, a method for improving synthetic accessibility of molecular structures may comprise generating a hierarchical library of molecule substructures representing possible molecular fragments. The method may further comprise collecting statistics on substructure occurrences in reference chemical datasets. The method may further comprise identifying synthetically irrelevant substructures based on the occurrence statistics, wherein low occurrence statistics may indicate the synthetically irrelevant substructures. The method may further comprise clustering and optimizing the library of irrelevant substructures. The method may further comprise filtering a group of molecular structures using a hierarchical algorithm to identify those containing irrelevant substructures. The method may further comprise removing the molecular structures containing irrelevant substructures from the group of molecular structures to obtain a filtered set of molecular structures that may be filtered by removing molecular structures having synthetically irrelevant substructures.

In some embodiments, a computer-implemented method for constructing a hierarchical library of molecular substructures for synthetic accessibility evaluation may comprise defining a zero layer comprising a core structure represented as a SMARTS string. The method may further comprise defining an element alphabet comprising atomic primitives for heteroatoms. The method may further comprise generating a first layer by combinatorially replacing generic atom symbols in the zero layer SMARTS string with atomic primitives from the element alphabet to produce heterocycle type substructures. The method may further comprise defining a substituent alphabet comprising bond primitives and atomic primitives for substituent types. The method may further comprise generating a second layer by replacing attachment point symbols in the first layer substructures with hydrogen atoms and substituent placeholders to produce position-specific substructures. The method may further comprise generating a third layer by enumerating substructures having two or three substituent positions. The method may further comprise generating a fourth layer by combinatorially substituting all substituent types from the substituent alphabet at each substituent position in the third layer substructures. The method may further comprise collecting occurrence statistics for each substructure in the fourth layer by querying a reference dataset of synthesized compounds. The method may further comprise identifying synthetically irrelevant substructures as those having occurrence frequency below a predetermined threshold. The method may further comprise storing the hierarchical library comprising the zero layer, first layer, second layer, third layer, and fourth layer with associated occurrence statistics.

In some embodiments, a computer-implemented method for filtering molecular structures generated by a generative chemistry model may comprise receiving a batch of molecular structures from a generative chemistry model, each molecular structure represented as a SMILES string. For each molecular structure in the batch, the method may comprise parsing the SMILES string to generate a molecular graph object. The method may further comprise applying a hierarchical filtering algorithm to determine whether the molecular graph object contains a synthetically irrelevant substructure by traversing a hierarchical library of SMARTS-encoded substructures. The method may further comprise assigning a PASS status when no synthetically irrelevant substructure may be detected. The method may further comprise assigning a FILTER status when a synthetically irrelevant substructure may be detected. The method may further comprise generating a filtered set comprising molecular structures having PASS status. The method may further comprise outputting the filtered set for synthesis prioritization.

In some embodiments, a computer-implemented method for selecting a synthetic route for a target molecule may comprise receiving a target molecule represented as a SMILES string. The method may further comprise generating a plurality of candidate synthetic routes for the target molecule. For each candidate synthetic route, the method may comprise identifying intermediate molecular structures. The method may further comprise applying a hierarchical filtering algorithm to each intermediate molecular structure to determine presence of synthetically irrelevant substructures. The method may further comprise calculating a synthetic accessibility score based on number of intermediate molecular structures containing synthetically irrelevant substructures. The method may further comprise selecting a preferred synthetic route having a lowest synthetic accessibility score. The method may further comprise outputting the preferred synthetic route for synthesis execution.

In some embodiments, a system for improving synthetic accessibility of molecular structures may comprise one or more processors. The system may further comprise memory storing instructions that, when executed by the one or more processors, may cause the system to perform the method of improving synthetic accessibility of molecular structures as described herein.

In some embodiments, a system for improving synthetic accessibility of molecular structures may comprise one or more processors. The system may further comprise memory storing instructions that, when executed by the one or more processors, may cause the system to perform the method of constructing a hierarchical library of molecular substructures as described herein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIGS. 1A-1B presents molecular structures generated by modern generative algorithms, which contain synthetically irrelevant substructures.

FIG. 2 includes the structural representation of the molecules with a conditional decomposition of molecular structures into rings (e.g., highlighted in gray), linkers (black), and peripheral fragments (gray, or terminal non-highlighted carboxylic acid, methoxy or fluorine).

FIG. 3 shows a comparison of molecular structure representations: SMILES (a) and SMARTS (b).

FIG. 4A includes a scheme for generating the hierarchical library of five-membered heterocycle substructures.

FIG. 4B includes a table showing the element alphabet of the heterocyclic core.

FIG. 4C shows Algorithm 1, which is a first layer generation.

FIG. 4D shows Algorithm 2, which is a second layer generation.

FIG. 4E shows Algorithm 3, which is a third layer generation.

FIG. 4F shows Algorithm 4, which is a fourth layer generation.

FIG. 4G shows Algorithm 5, which is a SMARTS clusterization.

FIG. 5 includes a visualization of a SMARTS substructure after substituent merging.

FIG. 6 shows an example computing device (e.g., a computer) that may be arranged in some embodiments to perform the methods (or portions thereof) described herein.

FIG. 7A show an example of the hierarchical filtering algorithm.

FIG. 7B shows Algorithm 6, which is for matching SMARTS with tree structure.

FIG. 8 includes a pie chart that illustrates the occurrence of substructures in the ChEMBL 29 database.

FIG. 9 illustrates an embodiment of the modules that are used for the synthetic accessibility modeling.

FIG. 10 illustrates a schematic representation of a complete hierarchical library of substructures that can be used to create representations of molecular structures.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology is related to a system with a computing device configured to execute drug design protocols using generative models, and generating thousands to millions of virtual structures. The computing protocols can prioritize the virtual structures, and molecules with structures having desired or defined characteristics can be selected for synthesis. Accordingly, the protocols can include synthesizing no more than a few dozen molecules from the virtual molecule structures that are generated, and thereby the synthetic route must be investigated, such as by synthetic accessibility. The present invention improves the synthetic accessibility systems and methods by using a model trained to identify synthetically irrelevant structures (e.g., including fragments or substructures that are not present in molecule databases) to filter out molecules that have the synthetically irrelevant structure. This strategy allows for the early elimination of molecular structures that raise even minimal concerns regarding synthetic feasibility based on local substructures, such as rings, linkers, or substituents within rings. If a structure contains even one synthetically irrelevant fragment (substructure), it can be discarded, as an alternative structure without such a synthetically irrelevant substructure can quickly be generated in its place. Thus, the present method for modeling synthetic accessibility exhibits extreme intolerance to false positives (e.g., falsely synthetically accessible results) while being significantly less sensitive to false negatives (e.g., falsely synthetically inaccessible results). Additionally, the high productivity of the generative model improves synthetic accessibility assessment methods in: computational efficiency, or the ability to process large volumes of data at high speed. Thus, providing an improvement to the technology.

A synthetically irrelevant-structure database can be created by systematically enumerating (or harvesting from generative outputs) molecules that contain at least one synthetically irrelevant fragment (i.e., a local substructure or junction environment that is absent or extremely rare in representative reference chemical spaces such as curated bioactivity databases or vendor screening collections). The database may be populated by (i) generating candidate molecules, (ii) decomposing each molecule into context-aware fragments (e.g., rings together with first-layer substituent environments), and (iii) labeling a molecule as “irrelevant” if any fragment fails a predefined occurrence threshold (for example, fragments observed fewer than a set number of times in the reference dataset), thereby yielding a high-precision set of negative examples enriched for “red-flag” motifs. This labeled corpus can then be used to train a filtering model, such as a conservative classifier operating on fragment fingerprints, SMARTS pattern hits, or hierarchical substructure features, to predict and reject molecules containing synthetically irrelevant fragments, with an explicit bias toward minimizing false positives (i.e., avoiding the acceptance of problematic structures). When deployed in a generative chemistry workflow, the resulting model acts as an early-stage gate: it rapidly screens newly generated molecules and filters out any structure that matches one or more synthetically irrelevant fragments, allowing the generator to replace them with close analogs that preserve design intent while staying within synthetically precedented chemical space.

To define synthetic accessibility (SA) properly, another important concept, synthetic feasibility (SF) can be defined first. Synthetic feasibility can be defined as the property of a molecule that can be either synthesizable (1) or not synthesizable (0), or in other words, synthetically feasible (1) or unfeasible (0), taking into account the whole current synthetic knowledge and technologies, all possible synthetic plans, all available starting materials, costs, labor and time that can be allocated for the synthetic campaign to get the molecule as a synthesized compound. Thus, SF can be considered as a Bernoulli random variable [15] with two possible outcomes: 1 and 0. Synthetic accessibility (SA) can be defined as p, which is a probability of success (1) for the Bernoulli random variable SF:

$$SF ( w ) = \{ \begin{matrix}0 , a compound is not synthesizable ; \\ 1 , a compound is synthesizable\end{matrix} .$$

$${F}_{SF} ( x ) = \{ \begin{matrix}0 , & if x < 0 ; \\ 1 - SA , & if 0 \leq x \leq 1 ; \\ 1 , & if x \geq 1\end{matrix} .$$

The complement of SA calculated as: q=1−p=1−SA

Here, q represents the probability of a compound not being synthesizable under current conditions (probability of failure, 0). This accounts for the inherent uncertainty and limitations in synthetic chemistry, reflecting the chance that, despite the theoretical accessibility, practical synthesis may not be achievable.

Since SA is defined as a Bernoullian probability (p), all properties and consequences of probability are true for SA, including the law of total probability:

$$SA = {\sum }_{i = 1}^{n} P ( S F = 1 \vert {C}_{i} ) \cdot P ( {C}_{i} ) ,$$

$$S {A}_{i} = P ( S F = 1 \vert {C}_{i} )$$

The SA accounts for multiple different countable (n) sets of conditions (C_i) in which condition-associated {SA}_i values can be calculated. Such conditions are different available sets of starting materials, synthetic plans to be assessed, synthetic methods and technologies available at a lab, labor, budget and time that can be allocated to the synthetic campaign, and the like.

Based on a probabilistic interpretation of synthetic accessibility, it is possible to identify factors that either increase or decrease the likelihood of successfully synthesizing a given molecular structure. For example, the existence of a plausible synthetic route and the availability of commercially accessible starting materials, while not guaranteeing success, can substantially improve the probability of completing a synthesis. Conversely, the presence of certain structural features may reduce the likelihood of a successful synthetic campaign, although their impact is context-dependent and not universally prohibitive.

Such features include, for instance, fragments containing asymmetric centers, which often necessitate the preparative separation of racemic mixtures or the use of stereoselective transformations to establish the desired configuration. These requirements introduce additional complexity and risk, including the possibility of failing to obtain the target enantiomer. Similarly, molecular structures containing macrocyclic motifs typically require macrocyclization reactions, which are frequently associated with low yields, challenging reaction conditions, and limited scalability. Another commonly cited risk factor is the presence of structural fragments that lack precedents in previously synthesized chemical space, which may serve as a practical “red flag” during compound selection for synthesis.

At the same time, it is important to emphasize that the absence of a given fragment from a reference dataset does not imply the fundamental impossibility of synthesizing a molecule containing it. Rather, the working hypothesis underlying many contemporary approaches to synthetic accessibility modeling is that molecules composed exclusively of fragments frequently observed in known, synthesized compounds are, on average, more likely to be synthetically accessible than those incorporating rare or previously unobserved fragments [16-21]. Methods based on this assumption are commonly classified as data-driven or statistical approaches to synthetic accessibility assessment.

These approaches have gained widespread adoption in modern Computer-Aided Drug Design (CADD), as their underlying rationale aligns closely with the practical and economic objectives of generative chemistry. Specifically, generative chemistry seeks to accelerate hit identification while minimizing cost and experimental risk, thereby favoring molecular structures with a higher estimated probability of successful synthesis and deprioritizing those deemed synthetically suspicious. Within this framework, a clear and operational definition of the term “fragment” becomes essential.

For example, the well-established SA Score [18] employs ECFC₄ fingerprints as its fundamental fragment representation. By performing statistical analysis over these fragments, the method estimates the relative “popularity” of local molecular environments across a reference chemical space. While this approach provides a convenient and computationally efficient proxy for synthetic accessibility, it also raises important conceptual concerns. In particular, circular fingerprints with a fixed radius of four bonds do not encode synthetic intent, reaction feasibility, or transformation logic, calling into question their suitability for accurately modeling the practical challenges of chemical synthesis.

In contrast, synthons or synthon-like fragments derived from retrosynthetic analysis offer a representation that is more closely aligned with chemical reasoning and synthetic practice. Such fragments reflect plausible disconnections and reaction steps, thereby providing a more interpretable and chemically meaningful basis for assessing synthetic accessibility. Furthermore, mapping these retrosynthetically derived synthons to their corresponding synthetic equivalents enables the identification of commercially available starting materials, directly linking fragment analysis to real-world synthesis planning.

The SA Score remains a canonical example of a first-order approximation to synthetic accessibility modeling. Owing to its simplicity, robustness, and low computational cost, it continues to be widely used in generative chemistry pipelines [22,23] and benchmarking platforms [24] for ranking candidate molecules and filtering out structures deemed synthetically infeasible.

Nevertheless, in certain cases, the SA Score and other widely used synthetic accessibility modeling approaches, including machine learning-based methods such as SCScore [25] and SYBA [19] may produce false positive predictions of synthetic feasibility. Such errors are particularly problematic in the context of rational molecular design using generative chemistry, where large numbers of candidate structures are produced and only a small subset can be evaluated experimentally. Therefore, in a synthetic accessibility module, it would be an improvement to inhibit false positive predictions of synthetic feasibility, which can be now obtained with the use of the hierarchical filtering module that filters out molecules with synthetically irrelevant structures or fragments. Thus, the synthetic accessibility module can be improved by including or being operably coupled with a hierarchical filtering module that is trained to filter out synthetically irrelevant structures.

In practical generative workflows, the presence of even a single synthetically irrelevant or highly problematic fragment within a molecular structure should typically result in its rejection, as structurally similar analogs lacking the problematic substructure can be readily generated as substitutes. Consequently, false positive predictions, such as molecules classified as synthetically accessible despite containing prohibitive structural features, are far more detrimental than false negatives, which merely exclude potentially synthesizable candidates.

Therefore, an effective synthetic accessibility model and modeling method can be explicitly biased toward minimizing false positives, exhibiting high intolerance to structures that are unlikely to be synthesizable in practice, even at the expense of increased false negatives. In other words, conservative rejection of questionable molecules is preferable to optimistic acceptance, as this strategy better aligns with the core objective of generative chemistry: to prioritize candidates with a high likelihood of successful synthesis and rapid experimental validation. Thus, the trained hierarchical filtering module that filters out synthetically irrelevant structures can improve the synthetic accessibility technology.

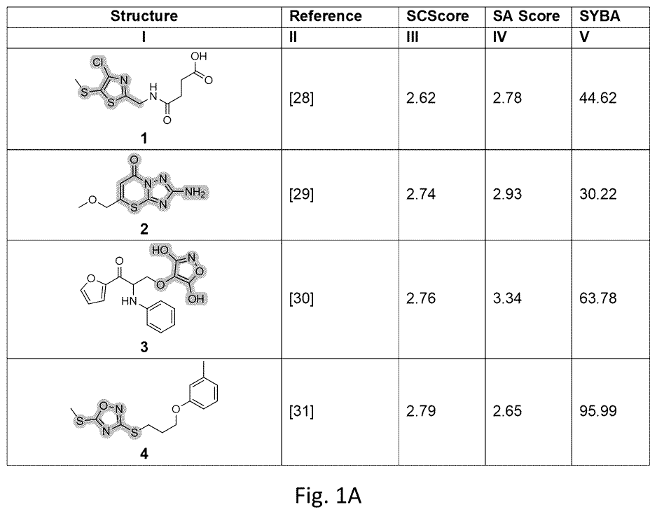

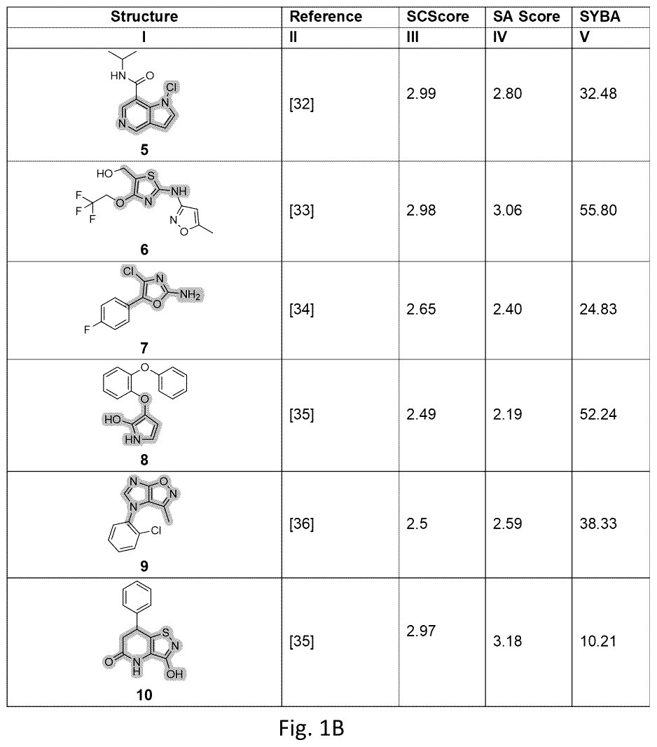

FIGS. 1A-1B presents molecular structures generated by modern generative algorithms, which contain synthetically irrelevant substructures. Based on the scores mentioned above, which are widely used synthetic accessibility modeling methods, all of these molecular structures would be classified as easily synthesizable, which is a false positive prediction. This is because each of the structures shown contains at least one synthetically irrelevant substructure (highlighted in gray). In this context, a synthetically irrelevant substructure refers to those fragments that cannot be found in chemical compounds represented in reference databases. Therefore, molecular structures containing these fragments should be filtered out, following the business logic of generative experiments described earlier.

FIGS. 1A-1B include a table of molecular structures generated by generative models containing at least one synthetically irrelevant fragment (highlighted in gray) and the results of synthetic accessibility modeling by SA Score, SCScore, and SYBA.

Additionally, optimized value ranges were used [19] to classify the presented molecular structures (see Table 1).

TABLE 1
Value ranges for SA Score, SCScore, and SYBA used
for the classification of molecular structures

| | | Recommended ranges | |
|---|---|---|---|
| | Value | for synthetically accessible | Optimized |
| Model | ranges | molecular structures | ranges [19] |
| SA Score | 1 ÷ 10 | 1 ÷ 6.5 | 1 ÷ 4.5 |
| SCScore | 1 ÷ 5 | — | 1 ÷ 3.1 |
| SYBA | (−∞; +∞) | [0; +∞) | [0; +∞) |

In some embodiments, the search for such suspicious substructures (e.g., having synthetically irrelevant fragment) can be performed manually or automatically using data from reference chemical spaces, such as public databases (e.g., ChEMBL [37], ZINC [38], or PubChem [39]) or catalogs from chemical compound suppliers (e.g., Enamine [40], WuXi [41]), or by utilizing commercial databases (e.g., SciFinder [42] or Reaxys [43]). In some aspects, such manual or automatically searching can be combined with the aforementioned high-throughput synthetic accessibility modeling methods, which can overcome problems with being insensitive to synthetically irrelevant substructures.

In medicinal chemistry, the use of substructural filters is a well-established practice for the early-stage prioritization of molecular structures. Such filters are commonly applied to exclude compounds containing toxophoric motifs or substructures associated with nonspecific binding, most notably those classified as Pan-Assay Interference Compounds (PAINS) [44]. As a standard workflow, medicinal chemists routinely subject chemical libraries and virtual screening databases to automated PAINS filtering prior to downstream analysis or experimental testing.

An analogous strategy could be employed for the assessment of synthetic accessibility, whereby molecular structures are efficiently filtered based on the presence of synthetically irrelevant or prohibitive substructures. Similar to PAINS filters, such a system can serve as a conservative, rule-based preselection tool, enabling the rapid exclusion of compounds that are unlikely to be synthetically viable in practice. Incorporating substructural filters for synthetic feasibility into generative chemistry pipelines would further reduce experimental risk and computational overhead by preventing the propagation of structurally problematic candidates at early stages of molecular design.

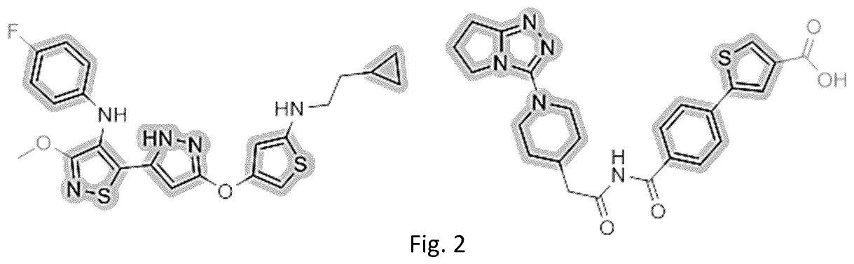

From the perspective of fragment composition, a molecular structure can be conditionally divided into the following elements: rings (e.g., saturated, unsaturated, aromatic, and macrocycles), linkers connecting the cyclic structures, and peripheral fragments (e.g., referred to as “decorations” in the literature). See the example in FIG. 2.

FIG. 2 includes the structural representation of the molecules with a conditional decomposition of molecular structures into rings (e.g., highlighted in gray), linkers (black), and peripheral fragments (gray, or terminal non-highlighted carboxylic acid, methoxy or fluorine).

In addition to synthesis feasibility of the ring or linker itself, synthetic accessibility can also consider the junction point where linkers connect to the ring, where the ring joins with peripheral fragments, as well as its local environment. Neither statistical methods for modeling synthetic accessibility, such as SA Score, nor machine learning-based methods explicitly take into account synthetically irrelevant environments at these junction points. For example, in the case of structure 1 from FIG. 1A, it is clear that while the individual components, such as thiazole and its substituents, are common, but the thiazole ring in this specific environment has not been encountered in reference databases.

As demonstrated in FIGS. 1A-1B, existing methods for modeling synthetic accessibility fail to filter molecular structures containing such synthetically irrelevant structural motifs. This highlights the need for the development of an algorithm capable of accounting for these features and deprioritizing such synthetically irrelevant structures. Based on the business paradigm of generative chemistry, which aims to reduce the cost of primary hit identification and the necessity for high algorithmic efficiency, it seems feasible to create an enumeration algorithm for such synthetically irrelevant substructures, using the occurrence statistics of these substructures in reference datasets.

In some embodiments, the protocol can be configured such that the less frequently a structural fragment appears in a dataset considered comprehensive, the higher the likelihood of a failed synthesis for the molecular structure containing this fragment. Such fragments are therefore synthetically irrelevant. This hypothesis is widely exploited in data-driven models of synthetic accessibility [16,18-20]. However, most often, the statistical contributions of individual fragments are summed, leading to the scenario illustrated in FIGS. 1A-1B.

In some embodiments, approaches to implement synthetically irrelevant screening can include: the creation and use of a set of allowed substructures based on the frequency (e.g., high frequency) of their occurrence in reference databases; the creation and use of a set of unallowed substructures based on the frequency (e.g., low frequency) of their occurrence in reference databases; and/or the creation and use of a set of substructures that are valency-appropriate, but synthetically irrelevant, as prohibitive filters. Therefore, a database of allowed substructures can be created. Additionally, a database of synthetically irrelevant substructures can be created. Each of the structures in these two databases can be used to train the hierarchical filtering model. In the first instance, the hierarchical filtering model becomes trained to filter out substructures based on frequency of fragments being less than a threshold value, such as number of such substructures in curated database of molecules, or less than a percentage. In the second instance, the hierarchical filtering model becomes trained to filter out substructures based on fragments that are synthetically irrelevant. Each approach has its advantages. In the first case, there is no need to develop a generative algorithm, but it requires the creation of a procedure to extract metadata for the first atom of substituents (e.g., charge, number of hydrogens attached, aromaticity, hybridization, number of neighboring heavy atoms, their type, etc.).

In some embodiments, the second approach is a problem of covering chemical space but appears to be more efficient because it could enable the creation of a high-performance filtering algorithm for irrelevant molecular structures, which can be quickly adapted to different reference chemical spaces as needed. As mentioned earlier, the presence of even one irrelevant substructure is sufficient grounds for rejecting a molecular structure and checking for such substructures is a routine cheminformatics task that can be halted as soon as the first substructure is detected. In contrast, checking all cycles in a structure for synthetic relevance requires verification of each cycle in the molecule. Given the need for high performance and the previously mentioned business logic, the present model and protocol will generate and use prohibitive filters for molecules or fragments that include a synthetically irrelevant structure or substructure.

### Protocols

To evaluate the proposed approach, five-membered aromatic heterocycles were selected as a model system due to their structural diversity and their prevalence in a wide range of bioactive compounds [45,46]. In addition, the extensive variety of synthetic strategies available both for constructing these heterocyclic cores and for introducing substituents at different positions makes them particularly challenging targets for assessing synthetic feasibility by visual inspection alone, especially when analyzing large sets of structures generated by generative models.

Importantly, synthetic accessibility is not determined solely by the feasibility of forming the heterocyclic core or linker itself. A critical determinant is the nature of the junctions at which substituents are attached to the heterocycle—specifically, the points where the core connects to peripheral fragments and the immediate chemical environment surrounding these connections. These junctions often dictate the choice of reactions, functional group compatibility, and overall robustness of the synthetic route.

To explicitly account for this aspect, the present study focuses on substructures derived from five-membered heterocycles that incorporate not only the heterocyclic ring itself but also its local environment, defined as the first layer of specified substituent atoms. By including this immediate substitution context, the resulting fragments more accurately capture synthetically relevant features associated with bond formation at the heterocycle-substituent interface.

In addition to five-membered aromatic heterocycles (e.g., a 5-membered aromatic heteroaryl ring selected from 2- or 3-furanyl, 2- or 3-thienyl, 2-, 3- or 4-pyrrolyl, imidazolyl, pyrazolyl, oxazolyl, isoxazolyl, thiazolyl and isothiazolyl), the approach described herein is equally applicable to a broader class of chemical moieties selected from the group consisting of six-membered aromatic heterocycles (including, but not limited to, pyridines, pyrimidines, pyrazines, triazines, and fused variants thereof), non-aromatic heterocycles of varying ring sizes, carbocyclic frameworks (including saturated, partially saturated, and bridged carbocycles), polycyclic and fused ring systems, aryl and heteroaryl groups, spirocyclic motifs, macrocyclic fragments, and highly substituted or densely functionalized ring systems. Further contemplated are moieties containing strained rings, multiple contiguous stereocenters, quaternary centers, unusual heteroatom arrangements, or uncommon bond types, as well as linker motifs and substituent patterns that impose challenging regio- or chemoselectivity constraints at points of attachment. As with five-membered heterocycles, substructures for these additional moieties may be defined to include the core scaffold together with its immediate local environment, comprising at least the first layer of substituent atoms and bonds at junction points, to capture synthetically relevant features that disproportionately influence feasibility. These Markush-listed classes encompass structural motifs that are frequently associated with increased synthetic complexity and therefore benefit from early, conservative filtration based on the presence of synthetically irrelevant or prohibitive substructures.

Based on the considerations outlined above, the present study proposes a systematic, fragment-centric framework for identifying and filtering synthetically irrelevant molecular substructures, with an initial focus on five-membered aromatic heterocycles, but the practical application can be to substantially any chemical motif. The core premise of this approach is that synthetic accessibility is strongly influenced not only by the identity of a molecular scaffold, but also by the immediate chemical environment at points of substitution and connectivity. Accordingly, the study is designed to explicitly capture and analyze these local environments in a statistically rigorous and chemically interpretable manner.

In a first step, a comprehensive fragment library is constructed that enumerates all possible five-membered aromatic heterocycles augmented with their local environments at linker attachment points. Each fragment in this library comprises the heterocyclic ring system together with the first layer of substituent atoms directly bonded to the ring. By incorporating this immediate substitution context, the resulting fragments encode synthetically relevant information about how the heterocycle is connected to the remainder of the molecular structure. This level of granularity is intended to capture junction-specific features, such as substitution patterns, heteroatom positioning, and bond types, that often dictate reaction feasibility, functional group compatibility, and the availability of viable synthetic transformations.

In the second step, occurrence statistics are collected for each heterocycle-environment combination (or other chemical moiety environment) using a curated database of real, experimentally synthesized compounds. For each fragment in the library, its frequency of appearance within known chemical space is quantified, thereby providing an empirical measure of how often a given local environment has been successfully realized in practice. These statistics serve as a proxy for synthetic precedent, reflecting not only the theoretical plausibility of a fragment but also its practical adoption in medicinal chemistry and related synthetic efforts. Importantly, this analysis does not assume that rarely observed fragments are inherently unsynthesizable; rather, it provides a probabilistic framework for distinguishing commonly realized motifs from those that are underrepresented and therefore potentially associated with increased synthetic risk.

Building on these occurrence statistics, the final step involves the development of an algorithmic filtering procedure to identify and exclude synthetically irrelevant or high-risk fragments. Fragments exhibiting low, negligible, or absent representation in the reference dataset are flagged as potentially problematic, particularly when their structural features are known to impose disproportionate synthetic complexity at points of attachment. The resulting filter is designed to operate conservatively, prioritizing the minimization of false positive predictions of synthetic accessibility. When integrated into generative chemistry workflows, this fragment-based filter enables the rapid rejection of candidate molecules containing such fragments, while allowing structurally similar alternatives with more favorable local environments to be generated in their place.

Taken together, this approach establishes a practical and interpretable mechanism for incorporating substructural synthetic knowledge into automated molecular design. By grounding synthetic accessibility assessment in fragment-level statistics derived from real chemical space, the proposed framework aligns with the operational needs of generative chemistry, where early-stage filtration of synthetically implausible structures is essential for reducing experimental risk, computational overhead, and overall discovery cost.

As a foundation for the proposed filtering algorithm of the hierarchical filtering model, hierarchically organized libraries are created of molecular substructures that explicitly account for the local chemical environment of each fragment. In the present implementation, this concept is applied to five-membered aromatic heterocycles; however, the approach is general and may be extended to other classes of molecular motifs. By “local environment,” the protocol refer to the immediate atomic and bonding context surrounding the core fragment, including the first layer of substituent atoms at linker attachment points. Capturing this local context is essential, as it is often the determining factor in whether a given fragment can be reliably incorporated into a larger molecular structure using feasible synthetic transformations.

The hierarchical organization of substructures plays a critical role in both computational efficiency and practical usability. Rather than treating each fragment as an independent pattern, fragments are arranged in a hierarchy that reflects increasing levels of structural specificity. More general parent fragments represent broad classes of substructures, while child fragments encode progressively more detailed local environments and substitution patterns. This hierarchical arrangement significantly reduces the computational cost of substructure searching within molecular graphs, as it allows rapid pruning of the search space by eliminating entire branches of the hierarchy when a parent pattern is absent. As a result, fragment matching can be performed efficiently even when screening large libraries of generated molecules.

For the formal representation of fragments within this hierarchical library, the SMARTS (SMILES Arbitrary Target Specification) language was selected. SMARTS provides a flexible yet concise means of encoding molecular patterns as text strings, making it particularly well suited for defining substructures and local environments in a machine-readable format. Unlike conventional SMILES, which are designed to describe specific, fully defined molecules, SMARTS enables the expression of generalized patterns through the use of atomic classes, bond specifications, and logical operators. This expressiveness is essential for representing families of related substructures within a single pattern definition.

A key advantage of SMARTS is its support for logical operators such as AND, OR, and NOT, which allows multiple related substructures to be combined into a single generalized representation. For example, while three distinct SMILES strings would be required to describe benzyl iodide, benzyl bromide, and benzyl chloride as individual molecules, an equivalent SMARTS pattern can be written to match all three benzyl halides simultaneously. In this way, SMARTS enables the definition of “superstructures” that subsume multiple chemically related motifs within a single fragment description. This capability is particularly advantageous for synthetic accessibility filtering, as it allows entire classes of synthetically challenging or undesirable motifs to be identified and excluded using compact and interpretable rules.

By combining hierarchical fragment organization with the expressive power of the SMARTS language, the proposed framework enables efficient, chemically meaningful substructure matching at scale. This design supports rapid identification of synthetically irrelevant or high-risk local environments during molecule generation and evaluation, while remaining transparent and interpretable to chemists. As such, it provides a practical bridge between data-driven statistical analysis and rule-based chemical intuition, reinforcing the conservative, false-positive-averse philosophy underlying the proposed synthetic accessibility filtering approach.

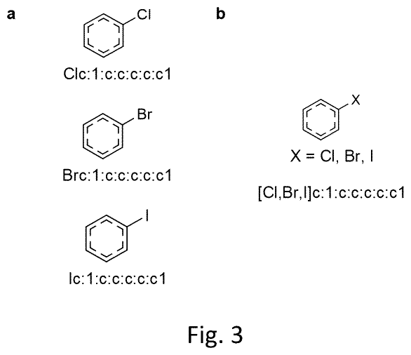

As the foundation for the filtering algorithm, hierarchically organized libraries of substructures are generated and saved (e.g., on a memory device) that take into account the local environment of the fragment, such as five-membered heterocycles or other chemical moieties. The hierarchical organization of fragments significantly reduces the time needed to search for a specific substructure in the molecular graph, thereby providing an improvement to the synthetical accessibility system. For fragment description, the SMARTS (SMILES arbitrary target specification [47]) language is used, which is the most flexible yet simple enough for representing molecular structures and fragments as strings. The SMARTS language allows the use of logical operators such as AND/OR. For example, to describe three benzyl halides (iodide, bromide, and chloride), one would need three separate SMILES strings [48], whereas SMARTS can express a superstructure combining all three substructures in a single line (see FIG. 3). FIG. 3 shows a comparison of molecular structure representations: SMILES (a) and SMARTS (b).

The ability to substantially reduce the number of distinct entries in a fragment library represents a key advantage when constructing large-scale substructure collections. By consolidating chemically related motifs into generalized representations, the overall size of the library can be minimized without sacrificing descriptive power. This reduction directly optimizes storage requirements, which becomes increasingly important as fragment libraries grow to encompass diverse scaffolds and local environments across broad regions of chemical space.

Beyond storage considerations, a smaller and more compact library significantly enhances the efficiency of substructure searching and filtering operations. Fewer entries translate into reduced pattern-matching overhead during molecular graph traversal, enabling faster identification of relevant substructures within candidate molecules. This efficiency gain is particularly critical in generative chemistry workflows, where large numbers of structures must be evaluated in real time or near real time. Therefore, it saves time, money and computing power to match synthetically irrelevant structures with rare fragments for a synthetic accessibility module.

As a result, the reduced and hierarchically organized fragment representation improves scalability, allowing synthetic accessibility filters to be applied consistently across extensive virtual libraries without becoming a computational bottleneck. This combination of compactness, search efficiency, and scalability further supports the practical integration of the proposed filtering framework into high-throughput molecular generation and screening pipelines, thereby improving synthetic accessibility modules. The ability to significantly reduce the number of entries is a major advantage when building large libraries. This reduction not only optimizes storage but also improves the efficiency of searching and filtering within the libraries, making the process of identifying relevant substructures faster and more scalable, and thereby an improvement to the synthetic accessibility technology.

The generation of a hierarchical library of SMARTS substructures, valid in terms of valency, was conducted by replacing symbols within the SMARTS string. This approach allows for minimal operations with molecular objects. The generation process was implemented in Python using built-in modules such as pandas, numpy, itertools, and the open-source cheminformatics package RDKit [49]. This created the database of synthetically irrelevant substructures (e.g., fingerprints, fragments, etc.).

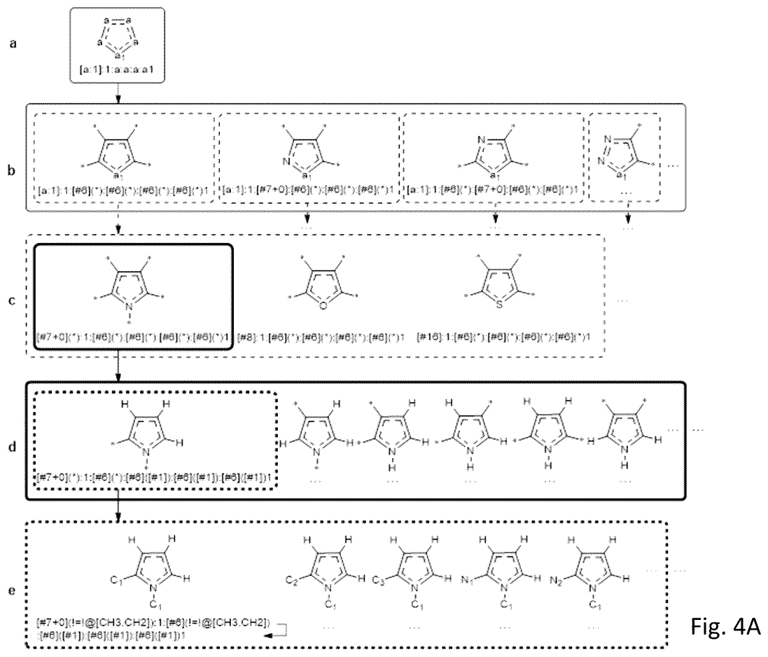

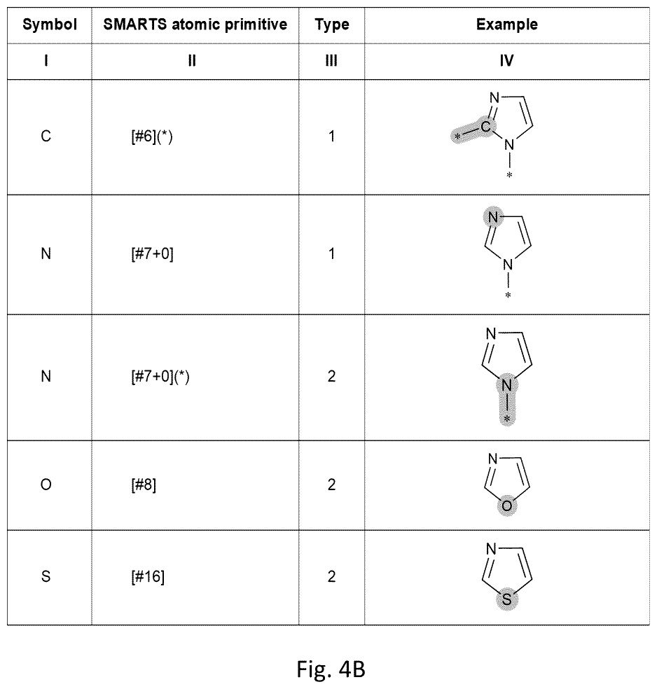

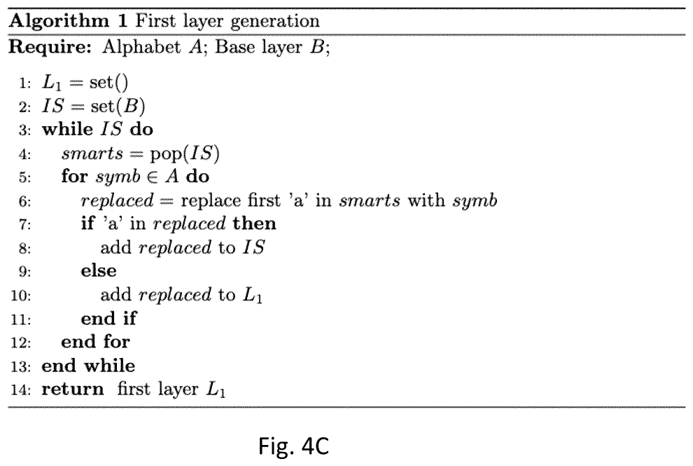

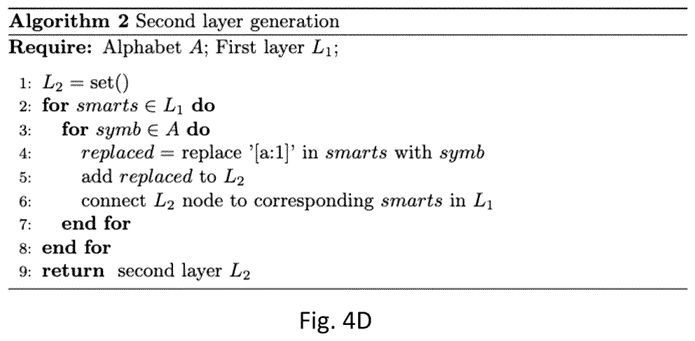

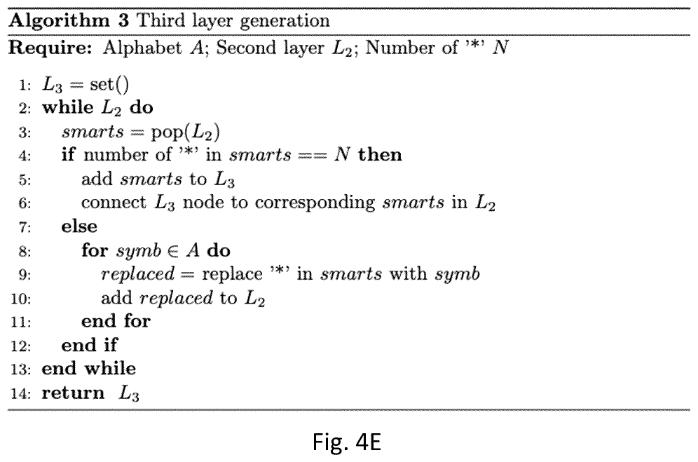

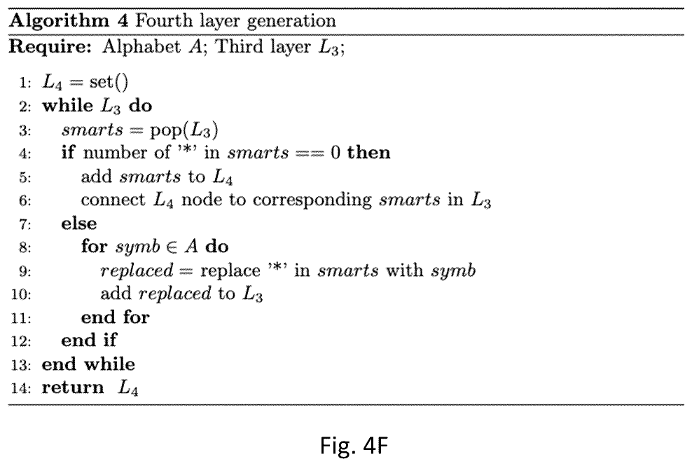

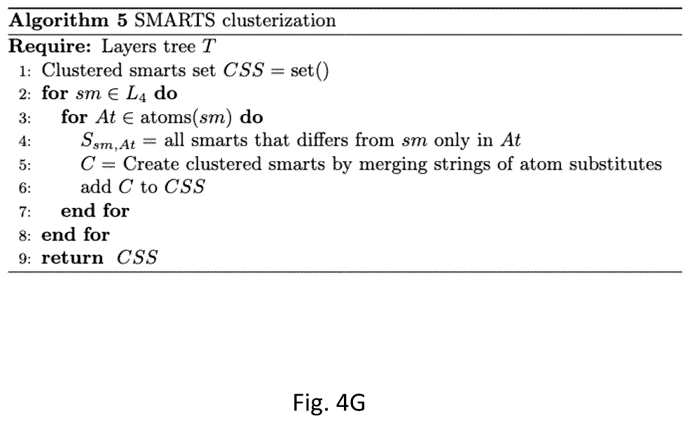

The first step involved defining the zero layer, such as a basic aromatic five-membered ring (FIG. 4A) (or other chemical moiety), and setting the element alphabet. For the proof of concept, the library was limited to five-membered heterocycles containing up to three heteroatoms. Thus, the alphabet consists of 5 elements (see FIG. 4B). It is also important to note that the element alphabet accounts for the “free valency” of an atom within the heterocycle, represented by an asterisk “*” if present. This is necessary so that the asterisk can later be replaced by a substituent atom or hydrogen.

FIG. 4A includes a scheme for generating the hierarchical library of five-membered heterocycle substructures.

FIG. 4B includes a table showing the element alphabet of the heterocyclic core.

FIG. 4C shows Algorithm 1, which is a first layer generation.

FIG. 4D shows Algorithm 2, which is a second layer generation.

By combinatorically replacing the symbol “a” in the zero-layer string with atomic primitives of Type 1 from the element alphabet, the first layer of the library is generated, which consists of 7 elements (see Algorithm 1 of FIG. 4C). Each element corresponds to a five-membered aromatic heterocycle with a specific number and position of heteroatoms (FIG. 4A, panel b).

Similarly, by replacing “[a:1]” with atomic primitives of type 2, the 3rd layer of the library was generated, containing 21 elements specific heterocycles (see Algorithm 2 of FIG. 4D and FIG. 4A, panel c). Since a single substructure can be represented by multiple different SMARTS strings, some substructures were duplicated. To identify and remove these duplicates, the SMARTS string was converted into an RDKit molecular object and the protocol performed an isomorphism check (using the standard HasSubstructMatch method from the RDKit library). If the molecular graphs matched completely, one of the duplicate strings was deleted.

The next step involves defining the substituents alphabet. The protocol identified 18 types of substituents, each represented by a single specified atom (see Table 2).

TABLE 2
Types of substituents used in the creation of the hierarchical library
of SMARTS substructures for five-membered heterocycles, considering
the local environment of the heterocycle. The heterocyclic core, relative
to which the substituent is considered, is highlighted in gray.

| | | SMARTS- | | |
|---|---|---|---|---|
| | SMARTS- | notation of | | |
| | notation | atom and its | | Functional |
| Symbol | of bond | environment | Description | group example |
| I | II | III | IV | V |
| C₁ | !=!@ | [CH3, CH2] | Aliphatic carbon with | —CH₃, —CH₂R |
| | | | two or three hydrogen | |
| | | | atoms | |
| C₂ | !=!@ | [CH, CH0] | Aliphatic carbon with | —CH(R)R, —C(R)(R)R, |
| | | | one hydrogen atom or | —C(O)R, —CN |
| | | | no hydrogens | |
| C₃ | !=!: | [c] | Aromatic carbon | |
| C₄ | !=@ | [C] | Aliphatic carbon in a | |
| | | | condensed ring | |
| C₅ | : | [c] | Aliphatic carbon in a | |
| | | | condensed ring | |
| N₁ | !=!@ | [NH2, NH, N+] | Aliphatic nitrogen with | —NH₂, —NHR, —NO₂, |
| | | | two or three hydrogen | —N⁺R₄ |
| | | | atoms, charged | |
| | | | aliphatic nitrogen | |
| N₂ | !=!@ | [N + 0H0] | Tertiary aliphatic | —N(R)R |
| | | | nitrogen | |
| N₃ | !=!: | [n + 0] | Aromatic nitrogen | |
| N₄ | : | [n + 0] | Nitrogen in an aromatic | |
| | | | condensed ring | |
| N₅ | !=@ | [N + 0] | Aliphatic nitrogen in a | |
| | | | condensed ring | |
| O₁ | !=!@ | [O] | Oxygen | —OH, —OR |
| O₂ | !=@ | [O] | Oxygen in a | |
| | | | condensed ring | |
| O₃ | : | [o] | Oxygen in an aromatic | |
| | | | condensed ring | |
| S₁ | !=!@ | [S] | Sulfur | —SH, —SR, —S(O)(O)R |
| S₂ | !=@ | [S] | Sulfur in a condensed | |
| | | | ring | |
| S₃ | : | [s] | Sulfur in an aromatic | |
| | | | condensed ring | |
| F | | F | Fluorine | —F |
| Cl | | Cl | Chlorine | —Cl |

The substituent is defined by both a bond primitive and an atomic primitive. This distinction is necessary to differentiate, for example, between C3 and C5, an aromatic carbon atom and an aromatic carbon atom in a fused ring system, which share the same atomic primitive but differ in their bonding context.

Next, by first replacing the “*” symbol with a hydrogen atom in the second-layer strings, and considering the accepted restriction of having 2 to 3 substituents in the substructure, the third layer is formed (see Algorithm 3, FIG. 4E). This layer consists of 86 elements and represents a set of second-layer heterocycles with two or three substituents, taking into account their positions (FIG. 4A panel d). After the combinatorial enumeration and substitution of all types of substituents (see Algorithm 4, FIG. 4F), 52,896 SMARTS substructures were generated, forming the fourth and final layer (FIG. 4A panel e). After removing duplicates (symmetrical substructures), 49,152 SMARTS strings remained.

Thus, a hierarchical library of substructures was built, where each layer represents a set of valid SMARTS strings, and each branch corresponds to the refinement of a substructure: from any five-membered aromatic ring at the zero layer to a specific heterocycle with specific types of substituents in specific positions at the fourth layer. The part of the library is shown in Table 3.

TABLE 3
The part of the structure of the hierarchical library of SMARTS
substructures for five-membered aromatic heterocycles

| Layer | ID | Description | SMARTS |
|---|---|---|---|
| I | II | III | IV |
| 0 | 0 | five-membered aromatic | [a:1]:1:a:a:a:a1 |
| | | heterocycle | |
| 1 | 1 | five-membered aromatic | [a:1]:1:[#6](*):[#6](*):[#6](*):[#6](*)1 |
| | | heterocycle with one | |
| | | heteroatom | |
| 1 | 2 | 1,2-azole | [a:1]:1:[#7+0]:[#6](*):[#6](*):[#6](*)1 |
| 1 | 3 | 1,3-azole | [a:1]:1:[#6](*):[#7+0]:[#6](*):[#6](*)1 |
| 1 | 4 | 1,2,3-diazole | [a:1]:1:[#7+0]:[#7+0]:[#6](*):[#6](*)1 |
| 1 | 5 | 1,2,4-diazole | [a:1]:1:[#7+0]:[#6](*):[#7+0]:[#6](*)1 |
| 1 | 6 | 1,2,5-diazole | [a:1]:1:[#7+0]:[#6](*):[#6](*):[#7+0]1 |
| 1 | 7 | 1,3,4-diazole | [a:1]:1:[#6](*):[#7+0]:[#7+0]:[#6](*)1 |
| 2 | 1.1 | pyrrole | [#7+0](*):1:[#6](*):[#6](*):[#6](*):[#6](*)1 |
| 2 | 1.2 | furan | [#8]:1:[#6](*):[#6](*):[#6](*): [#6](*)1 |
| 2 | 1.3 | thiophene | [#16]:1:[#6](*):[#6](*):[#6](*):[#6](*)1 |
| 2 | 2.1 | pyrazole | [#7+0](*):1:[#7+0]:[#6](*):[#6](*):[#6](*)1 |
| 2 | 2.2 | isoxazole | [#8]:1:[#7+0]:[#6](*):[#6](*):[#6](*)1 |
| 2 | 2.3 | isothiazole | [#16]:1:[#7+0]:[#6](*):[#6](*):[#6](*)1 |
| 2 | . . . | . . . | . . . |
| 3 | 1.1.1 | 1,2-disubstituted pyrrole | [#7+0](*):1:[#6](*):[#6]([#1]):[#6]([#1]):[#6]([#1])1 |
| 3 | 1.1.2 | 1,3-disubstituted pyrrole | [#7+0](*):1:[#6]([#1]):[#6](*):[#6]([#1]):[#6]([#1])1 |
| 3 | 1.1.3 | 2,3-disubstituted pyrrole | [#7+0]([#1]):1:[#6](*):[#6](*):[#6]([#1]):[#6]([#1])1 |
| 3 | 1.1.4 | 2,4-disubstituted pyrrole | [#7+0]([#1]):1:[#6](*):[#6]([#1]):[#6](*):[#6]([#1])1 |
| 3 | . . . | . . . | . . . |
| 3 | 1.1.7 | 1,2,3-trisubstituted pyrrole | [#7+0](*):1:[#6](*):[#6](*):[#6]([#1]):[#6]([#1])1 |
| 3 | . . . | . . . | . . . |
| 3 | 1.2.1 | 2,3-disubstituted furan | [#8]:1:[#6](*):[#6](*):[#6]([#1]):[#6]([#1])1 |
| 3 | 1.2.2 | 2,4-disubstituted furan | [#8]:1:[#6](*):[#6]([#1]):[#6](*):[#6]([#1])1 |
| 3 | . . . | . . . | . . . |
| 3 | 2.1.1 | 1,3-disubstituted pyrazole | [#7+0](*):1:[#7+0]:[#6](*):[#6]([#1]):[#6]([#1])1 |
| 3 | 2.1.2 | 1,4-disubstituted pyrazole | [#7+0](*):1:[#7+0]:[#6]([#1]):[#6](*):[#6]([#1])1 |
| 3 | . . . | . . . | . . . |
| 4 | 1.1.1.1 | 1,2-C1-disubstituted | [#7+0](!=!@[CH3,CH2]):1:[#6](!=!@[CH3,CH2]) |
| | | pyrrole | :[#6]([#1]):[#6]([#1]):[#6]([#1])1 |
| 4 | 1.1.1.2 | 1-C1-2-C2-substituted | [#7+0](!=!@[CH3,CH2]):1:[#6](!=!@[CH,CH0]) |
| | | pyrrole | :[#6]([#1]):[#6]([#1]):[#6]([#1])1 |
| 4 | 1.1.1.3 | 1-C1-2-C3-substituted | [#7+0](!=!@[CH3,CH2]):1:[#6](!=!:[c]):[#6]([#1]) |
| | | pyrrole | :[#6]([#1]):[#6]([#1])1 |
| 4 | . . . | . . . | . . . |
| 4 | 1.1.2.1 | 1,3-C1-disubstituted | [#7+0](!=!@[CH3,CH2]):1:[#6]([#1]):[#6](!=! |
| | | pyrrole | @[CH3,CH2]):[#6]([#1]):[#6]([#1])1 |
| 4 | 1.1.2.2 | 1-C1-3-C2-substituted | [#7+0](!=!@[CH3,CH2]):1:[#6]([#1]):[#6](!=! |
| | | pyrrole | @[CH,CH0]):[#6]([#1]):[#6]([#1])1 |
| 4 | . . . | . . . | . . . |
| 4 | 1.1.7.1 | 1,2,3-C1-trisubstituted | [#7+0](!=!@[CH3,CH2]):1:[#6](!=!@[CH3,CH2]) |
| | | pyrrole | :[#6](!=!@[CH3,CH2]):[#6]([#1]):[#6]([#1])1 |
| 4 | 1.1.7.2 | 1-C1-2-C1-3-C2- | [#7+0](!=!@[CH3,CH2]):1:[#6](!=!@[CH3,CH2]) |
| | | substituted pyrrole | :[#6](!=!@[CH,CH0]):[#6]([#1]):[#6]([#1])1 |
| 4 | 1.1.7.3 | 1-C1-2-C1-3-C3- | [#7+0](!=!@[CH3,CH2]):1:[#6](!=!@[CH3,CH2]) |
| | | substituted pyrrole | :[#6](!=!@[c]):[#6]([#1]):[#6]([#1])1 |
| 4 | . . . | . . . | . . . |
| 4 | 2.1.1.1 | 1,3-C1-disubstituted | [#7+0](!=!@[CH3,CH2]):1:[#7+0]:[#6](!=!@ |
| | | pyrazole | [CH3,CH2]):[#6]([#1]):[#6]([#1])1 |
| 4 | 2.1.1.2 | 1-C1-3-C2-substituted | [#7+0](!=!@[CH3,CH2]):1:[#7+0]:[#6](!=!@ |
| | | pyrazole | [CH,CH0]):[#6]([#1]):[#6]([#1])1 |
| 4 | . . . | . . . | . . . |
| 4 | 2.1.2.1 | 1,4-C1-disubstituted | [#7+0](!=!@[CH3,CH2]):1:[#7+0]:[#6]([#1]):[#6] |
| | | pyrazole | (!=!@[CH3,CH2]):[#6]([#1])1 |
| 4 | 2.1.2.2 | 1-C1-4-C2-substituted | [#7+0](!=!@[CH3,CH2]):1:[#7+0]:[#6]([#1]):[#6] |
| | | pyrazole | (!=!@[CH,CH0]):[#6]([#1])1 |
| 4 | . . . | . . . | . . . |

### Collection of Statistics for the Library

As reference sets of synthesized compounds, the following were used: open ChEMIBL 29 database [50] with 1,850,431 unique structures; and the commercially available screening compound database from Enamine [40](HTS, Advanced, Premium Collections) 2,823,257 unique compounds after standard preprocessing (structure standardization, removal of salt components, removal of duplicates). The analysis of substructure occurrence statistics was performed independently for both datasets using the standard isomorphism procedure (Table 4).

TABLE 4
Number of substructure occurrences in the training sets

| Number of occurrences | ChEMBL 29 | Enamine |
|---|---|---|
| ≥1 | 5,023 | 3,069 |
| ≥10 | 2,440 | 1,891 |
| <10 | 46,712 | 47,261 |

As a fundamental hypothesis regarding the nature of synthetic accessibility, it is proposed that the presence of a rare substructure in a molecular structure decreases the likelihood that the molecule is synthetically feasible. A substructure occurrence frequency threshold of 10 was selected, as substructures appearing less frequently may represent rare, atypical structures, which could raise concerns about their synthetic accessibility and realism. This threshold helps to exclude potentially erroneous or artifact substructures. However, any other reasonable threshold integer could be used, such as 8-15 or 7-20, or the like.

### Library Optimization

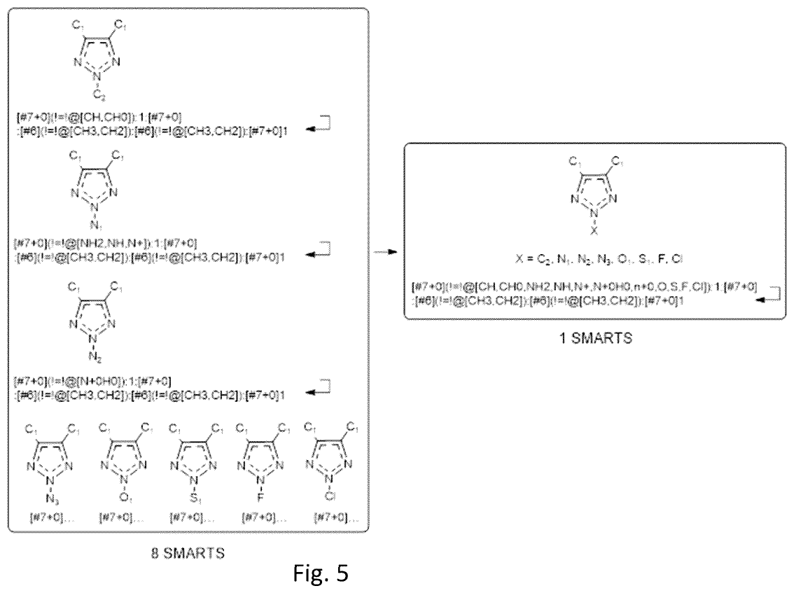

Due to the large number of penalizing fragments in their SMARTS strings, homogeneous substituents were combined automatically (see Algorithm 5 in FIG. 4G). This procedure reduced the number of penalizing SMARTS substructures from 46,250 to 7,157. An example of a combined SMARTS substructure is shown in FIG. 5. In the most successful cases, up to 12 substructures were merged into a single SMARTS string. This process is called clusterization by analogy with the process of combining several objects into one. It is worth noting that similar operations on SMARTS strings in an automated mode had not been previously described in the relevant literature.

FIG. 5 includes a visualization of a SMARTS substructure after substituent merging.

In some embodiments, increasing the complexity of the SMARTS object has little to no impact on the time required for the isomorphism procedure. The majority of the time is spent on graph matching, while the comparison of atomic primitives from the list is executed almost instantaneously.

### Hierarchical Filtering Algorithm

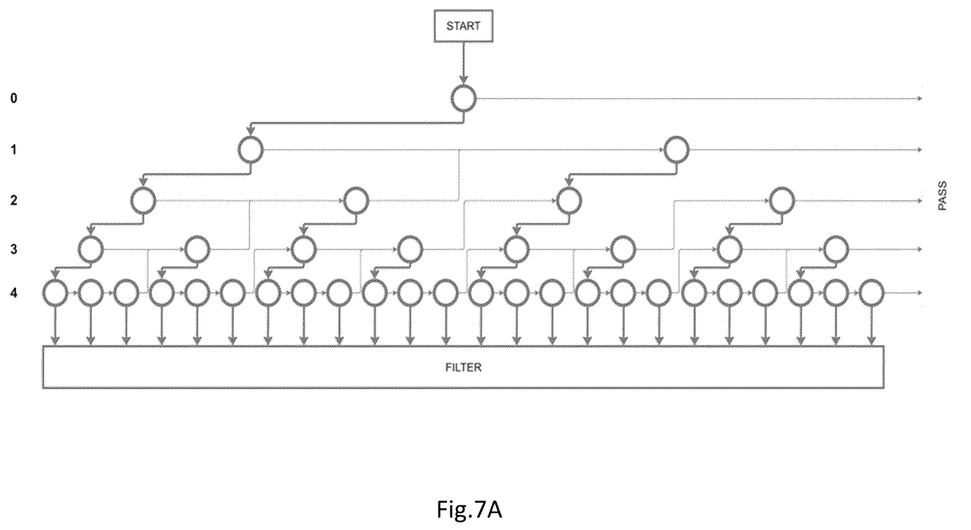

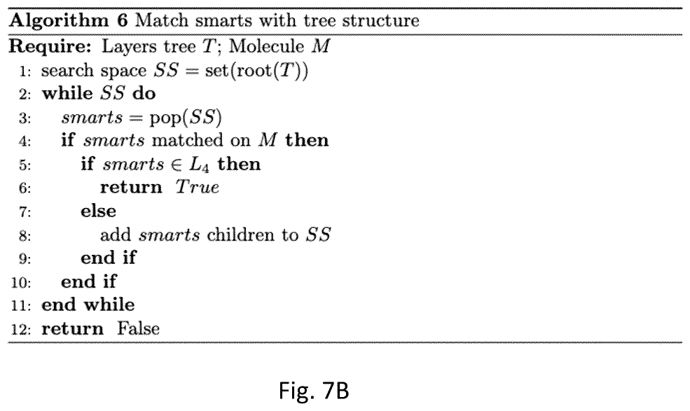

The hierarchical structure of the penalizing SMARTS substructure library enabled the development of a hierarchical filtering algorithm (e.g., hierarchical filtering model) for searching these substructures in molecular structures. The algorithm is a classic tree search, which terminates upon finding the first substructure from the final layer of the library (see FIG. 7A and Algorithm 6 of FIG. 7B). The input is a SMILES string. Initially, the presence of a five-membered aromatic heterocycle is checked; if absent, the algorithm terminates, and the molecular structure passes the filter. If the heterocycle is detected, the algorithm checks for the presence of the first substructure from the first layer of the library. If a match is found, the search continues to the next layer (on the fourth and final layer, the structure is rejected, and the algorithm terminates); if no match is found, the algorithm moves to the next substructure in the same layer (switching branches). If no substructures from the layer are detected (e.g., the five-membered aromatic heterocycle does not match any substructures from the library), the algorithm terminates without applying a penalty. This molecule is then passed and may be not be filtered out by the hierarchical filtering algorithm.

FIG. 7A includes a scheme of the hierarchical filtering algorithm. Each node corresponds to executing a subgraph isomorphism search procedure for the corresponding substructure from the library (default HasSubstructMatch method of RDKit). In the case of success (substructure found), the algorithm proceeds to the next layer (bold gray arrow). Otherwise, the search moves to the neighboring node or the next node in the previous layer (thin gray arrow). Reaching the rectangle labeled “FILTER” indicates that the molecular structure has failed the filtration and contains a synthetically irrelevant fragment while reaching the rectangle labeled “PASS” suggests that the molecular structure has successfully passed the filter

This approach, combined with reducing the library through the merging of homogeneous substituents, significantly increased (by more than 250 times in the same conditions) the performance of molecular structure filtering compared to the sequential enumeration algorithm (Table 5). The achieved performance is more than sufficient for filtering molecules generated by modern generative models.

TABLE 5
Comparison of filtering algorithm performance for synthetically irrelevant molecular
structures on a set of 30,000 compounds generated by the GENTRL model [31]

| | | | Hierarchical |
|---|---|---|---|
| | Sequential | Hierarchical | enumeration of |
| Algorithm | enumeration | enumeration | clusterized library |
| Number of SMARTS- | 46,250 | 46,250 | 7,157 |
| strings to match | SMARTS-strings | SMARTS-strings | SMARTS-strings |
| Time of algorithm | 1:22:45 | 0:00:50 | 0:00:20 |
| execution on 1 CPU, | | | |
| hh:mm:ss | | | |
| Algorithm performance | 6.04 | 600 | 1,500 |
| on 1 CPU, structures/s | | | |

Although it may seem that the clusterization of SMARTS objects did not show impressive results in terms of speedup (the speedup compared to the non-clustered library was only 2.5 times, see Table 7), the inventors have invented an approach that can significantly speed up the substructure search algorithm for larger substructure libraries, which is a technical improvement.

### Experimental

### Substructure Library Analysis

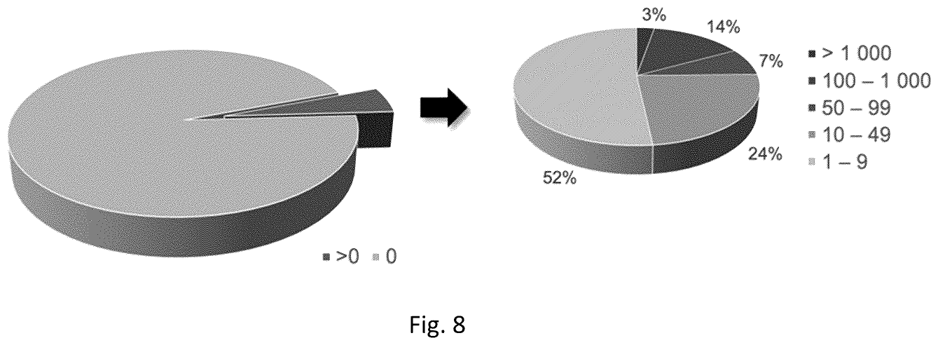

Only about 10% (5,023) of the generated substructures are found in the real chemical space of ChEMBL 29. Among these, more than 50% are observed between 1 and 9 times, and only 3% (0.3% of the total number of fragments in the library) are detected more than 1,000 times (FIG. 8). Notably, around 90% of the substructures (44,129) were never found in the ChEMBL 29 database.

FIG. 8 includes a pie chart that illustrates the occurrence of substructures in the ChEMBL 29 database. The pie chart on the left shows substructures that were never found in the database in gray, while substructures that were found at least once are other shades. The pie chart on the right provides a breakdown into different classes based on their frequency of occurrence.

Most commonly among five-membered aromatic heterocycles in the ChEMBL 29 database, the substructures corresponding to indoles and imidazopyridines are found, while in the Enamine set, substructures of pyrazoles and oxadiazoles prevail. This can be explained by the differences in the purpose of the databases: ChEMBL 29 is a collection of bioactive molecules that includes many natural compounds and their analogs, whereas the Enamine collection consists of specially synthesized compounds aimed at high-throughput screening tasks in accordance with current trends in drug design. Thus, the developed approach can serve not only as a tool for modeling the synthetic accessibility of molecules but also for analyzing trends, identifying privileged fragments in molecule sets, and as a guide for generative models.

### Analysis of Molecular Structures Generated by Generative Models

The developed algorithm effectively filters out molecular structures 1-10, as shown in the tables of FIGS. 1A-1B, due to the presence of substituted five-membered aromatic heterocycles that do not appear in the training dataset.

As an additional experiment, an assessment was conducted on the number of synthetically irrelevant molecular structures according to the developed algorithm applied to the outputs of existing generative models. The results of the analysis of the generations from the generative chemistry platform Chemistry42 are presented in Tables 6-7. It is shown that the proportion of penalized structures ranges from 3% to 39% for different targets. Notably, all structures generated by the Chemistry42 platform, by default, meet the criterion for synthetic accessibility, which is modeled on the platform using the ReRSA algorithm, further emphasizing the relevance of the developed algorithm for filtering structures containing synthetically irrelevant substructures.

TABLE 6
Results of the analysis of the outputs from the Chemistry42 generative platform [51]

| | Number of generated | Number of filtered |
|---|---|---|
| Name of the generation experiment | molecular structures | molecular structures |
| I | II | III |
| Design of Janus kinase 3 (JAK3) inhibitors based | 4,419 | 1,296 (29%) |
| on target structure | | |
| Design of ubiquitin-specific processing protease 7 | 4,378 | 425 (10%) |
| (USP7) inhibitors based on target structure using | | |
| privileged substructures | | |
| Design of epidermal growth factor receptor | 2,812 | 905 (32%) |
| (EGFR) tyrosine kinase inhibitors based on target | | |
| structure using pharmacophore hypotheses | | |
| Scaffold-hopping of the known calcium/calmodulin- | 1,512 | 90 (6%) |
| dependent protein kinase 2 (CAMKK2) inhibitor | | |
| Optimization of the lead compound, an inhibitor | 7,334 | 252 (3%) |
| of MPS-1 kinase | | |
| Fragment-based design of non-covalent inhibitors | 1,968 | 120 (6%) |
| of the main protease of the SARS-CoV-2 | | |

TABLE 7
Results of the analysis of the outputs of generations with fixed aromatic
five-membered ring from the Chemistry42 generative platform

| | Number of generated | Number of filtered |
|---|---|---|
| Name of the generation experiment | molecular structures | molecular structures |
| Design of phosphoinositide 3-kinase (PI3K) | 5,102 | 1,994 (39%) |
| inhibitors, analogs of alpelisib, based on target | | |
| structure. | | |
| Design of ABL2 tyrosine kinase inhibitors, analogs | 2,566 | 706 (28%) |
| of dasatinib, based on target structure | | |
| Design of RET tyrosine protein kinase inhibitors, | 3,992 | 1,464 (37%) |
| analogs of selpercatinib, based on target structure | | |

Generations from Chemistry42 with a fixed five-membered aromatic fragment were also analyzed (see Table 7). These conditions require that each generated molecular structure contain a five-membered aromatic heterocycle. It was shown that, in this case, the proportion of structures filtered out by the developed algorithm increases, ranging from 28% to 39% for different targets.

In addition to the mentioned experiments, published datasets of molecular structures generated by various generative models over the past 6 years were analyzed (see Table 8). It is important to note that the molecular structures were evaluated solely in terms of their validity and synthetic accessibility in the context of five-membered aromatic heterocycles without assessing their compliance with drug-like criteria. The proportion of filtered molecular structures ranged from 0.3% to 28.5%. Notably, the synthetic generator SynthI, despite its nature and purpose, produced approximately 5% synthetically irrelevant structures. These experiments underscore the relevance of the developed method for modeling synthetic accessibility.

TABLE 8
Results of the analysis of outputs from modern generative models

| | | Number of | | |
|---|---|---|---|---|
| | | structures in | Number | Number |
| | | the published | of invalid | of filtered |
| No | Publication title | dataset | structures | structures |
| I | II | III | IV | V |
| 1 | Generating Focused Molecule Libraries | 454,700 | 619 | 6,614 |
| | for Drug Discovery with Recurrent Neural | | | (1.46%) |
| | Networks [28] | | | |
| 2 | Deep learning enables rapid | 30,000 | 0 | 1,933 |
| | identification of potent DDR1 kinase | | | (6.44%) |
| | inhibitors [31] | | | |
| 3 | Shape-Based Generative Modeling for | 2,106,904 | 0 | 4,202 |
| | de Novo Drug Design [29] | | | (0.20%) |
| 4 | Cov_FB3D: A De Novo Covalent Drug | 220,377 | 1 | 14,433 |
| | Design Protocol Integrating the BA- | | | (6.55%) |
| | SAMP Strategy and Machine-Learning- | | | |
| | Based Synthetic Tractability Evaluation | | | |
| | [52] | | | |
| 5 | De novo design of new chemical entities | 1,267 | 0 | 21 |
| | for SARS-CoV-2 using artificial | | | (1.66%) |
| | intelligence [53] | | | |
| 6 | Accelerating De Novo Drug Design | 6,106 | 0 | 158 |
| | against Novel Proteins Using Deep | | | (2.59%) |
| | Learning [36] | | | |
| 7 | Deep Learning-Based Ligand Design | 3,183 | 0 | 50 |
| | Using Shared Latent Implicit Fingerprints | | | (1.57%) |
| | from Collaborative Filtering [54] | | | |
| 8 | Automated Generation of Novel | 188 | 0 | 9 |
| | Fragments Using Screening Data, a Dual | | | (4.79%) |
| | SMILES Autoencoder, Transfer Learning | | | |
| | and Syntax Correction [55] | | | |
| 9 | Transformer-Based Generative Model | 7,052 | 0 | 68 |
| | Accelerating the Development of Novel | | | (0.96%) |
| | BRAF Inhibitors [56] | | | |
| 10 | Artificial Intelligence-Guided De Novo | 200 | 0 | 57 |
| | Molecular Design Targeting COVID-19 | | | (28.50%) |
| | [30] | | | |
| 11 | Kinase Inhibitor Scaffold Hopping with | 381,579 | 32,516 | 4,386 |
| | Deep Learning Approaches [57] | | | (1.26%) |
| 12 | De Novo Structure-Based Drug Design | 20,000 | 1 | 447 |
| | Using Deep Learning [34] | | | (2.24%) |
| 13 | DeLA-Drug: A Deep Learning Algorithm | 15,000 | 42 | 258 |
| | for Automated Design of Druglike | | | (1.72%) |
| | Analogues [32] | | | |
| 14 | An In Silico Explainable Multiparameter | 7,080 | 0 | 21 |
| | Optimization Approach for De Novo Drug | | | (0.30%) |
| | Design against Proteins from the Central | | | |
| | Nervous System [35] | | | |
| 15 | Synthl: A New Open-Source Tool for | 12,060,315 | 0 | 559,942 |
| | Synthon-Based Library Design [58] | | | (4.64%) |
| 16 | De novo design of anti-tuberculosis | 4,041 | 0 | 50 |
| | agents using a structure-based deep | | | (1.24%) |
| | learning method [33] | | | |

In this experiment, invalid structures did not pass the standard validation and normalization procedure of RDKit. It is important to note that the molecular structures were evaluated solely in terms of their validity and synthetic accessibility in the context of five-membered aromatic heterocycles without assessing their compliance with drug-like criteria.

### Synthetically Irrelevant Filtering Module

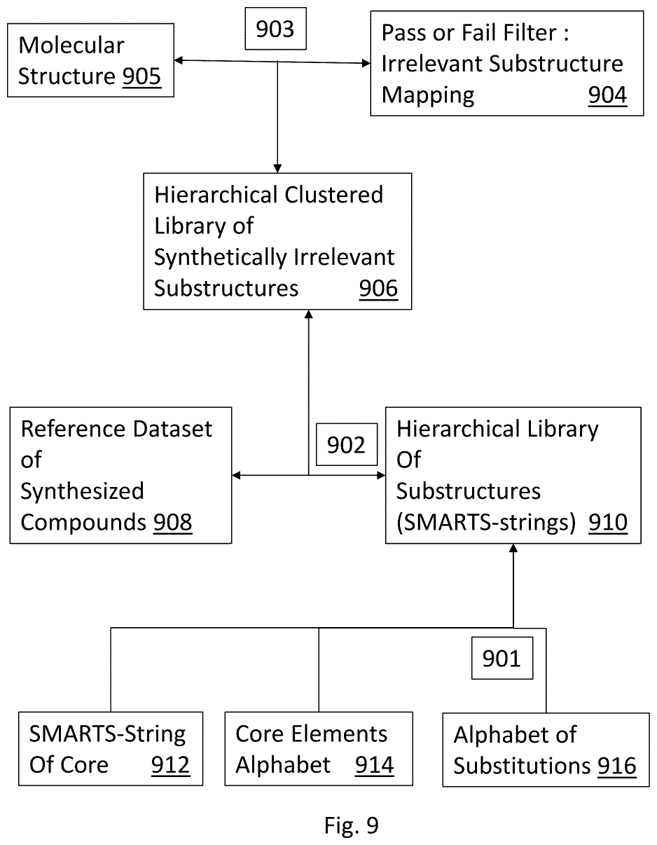

FIG. 9 illustrates an embodiment of the modules that are used for the synthetic accessibility modeling. FIG. 9 illustrates the principal schema of the developed approach for modeling synthetic accessibility based on filtering structures containing synthetically irrelevant substructures.

The system consists of several blocks. The core block is the step of creating a hierarchically organized library (block 910) of all possible SMARTS substructures from a predefined core string (block 912) and alphabets of core elements (block 914) and alphabet of substitutions (block 916) (see: procedure 901 on FIG. 9). Next, the process of collecting statistics for substructure occurrences in the reference chemical space (block 908) is applied (procedure 902). This step allows the identification of synthetically irrelevant substructures to be filtered. After identifying such substructures, the algorithm of clusterization of homogeneous SMARTS strings is applied. This is the final step of precomputations, and the result of these procedures is the hierarchical clustered library of synthetically irrelevant substructures (block 906). This library is used for filtering molecular structures (block 905) containing synthetically irrelevant substructures via a hierarchical filtering algorithm (procedure 903). As a result of applying this filtering algorithm to a molecular structure, the boolean value is returned: TRUE or FALSE, which means the structure passed or violated the filtering system (block 904). Also, if the structure violated the filtering systems, that means that it contains synthetically irrelevant substructure, and the substructure's atom mappings are returned. It can be used for the visualization of the problem substructure.

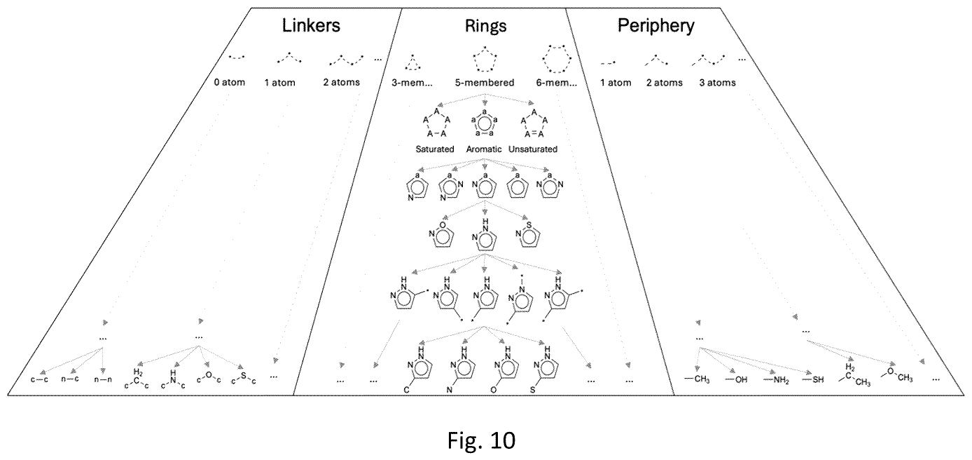

In summary, the developed approach for creating hierarchically organized libraries of substructures can be applied to any type of substructure, such as cyclic (as demonstrated using the example of aromatic five-membered heterocycles) and acyclic (linkers and periphery), such as in FIG. 10. Such a comprehensive system would enable the description of any molecular structure and essentially represent a new form of structural representation, where fingerprints of molecular structures could be constructed based on the tree layers (e.g., binary strings or matrixes where one bit or element of the matrix is associated with the presence of one of the substructures). The hierarchical organization of the system would allow flexible control over the level of abstraction, which could be particularly useful, for instance, in developing a new similarity metric.

FIG. 10 illustrates a schematic representation of a complete hierarchical library of substructures that can be used to create representations of molecular structures.

In certain embodiments, the present invention provides a computer-implemented method for representing molecular structures using a hierarchically organized library of SMARTS-encoded substructures that explicitly incorporate the local chemical environment of each fragment. The hierarchical substructure representation enables systematic decomposition of a molecular structure into context-aware fragments corresponding to core motifs and their immediate substituent environments, thereby facilitating efficient structural analysis and comparison.

In this work, we proposed a new approach to represent a molecular structure using the hierarchically organized library of SMARTS substructures that take into account the local environment of the fragment. The developed approach allows for multiple applications, including synthetic accessibility analysis based on the filtering of molecular structures that contain synthetically irrelevant substructures. The main idea of the method was inspired by the common hypothesis that if a molecule contains only structural motifs that are commonly found in reference chemical space, it is likely to be easier to synthesize than one that does not contain such structural motifs.

In some embodiments, the hierarchical library is employed to evaluate synthetic accessibility of molecular structures by identifying and filtering compounds that contain one or more synthetically irrelevant substructures. The method operates by matching substructures of an input molecular structure against the hierarchical library and determining whether the structure satisfies predefined acceptability criteria. Molecular structures containing substructures classified as synthetically irrelevant are rejected or deprioritized, while structures composed predominantly or exclusively of acceptable substructures are retained for further consideration.

In further embodiments, the filtering process is based on the principle that molecular structures composed of structural motifs frequently observed in a reference chemical space of synthesized compounds are more likely to be synthetically tractable than structures containing rare, unobserved, or atypical motifs. By encoding this principle in the hierarchical SMARTS library, the invention provides a conservative, rule-based mechanism for assessing synthetic feasibility that is particularly well suited for integration into generative chemistry and computer-aided molecular design workflows.

In additional embodiments, the described representation and filtering approach may be applied to a variety of downstream tasks, including but not limited to hit prioritization, virtual library curation, de novo molecule generation, and early-stage compound triage, thereby reducing computational overhead, experimental risk, and development cost associated with the synthesis of chemically impractical structures.

In certain embodiments, the algorithms disclosed herein are extensible and may be applied to a broad range of molecular substructures, including cyclic and acyclic fragments. The invention is not limited to any particular class of chemical motifs, and it is contemplated that a detailed hierarchical library may be constructed for any selected fragment class, including but not limited to heterocycles, carbocycles, linkers, functional groups, and substituent patterns of varying complexity.

The algorithms developed to substantiate the approach can be extended for application to any types of substructures cyclic and acyclic. It is implied that a detailed hierarchical library can be created for any class of fragments, and statistics can be collected based on any dataset or combination of datasets, whether open or proprietary. The list of substituents can also be expanded and revised, including substituents with descriptions of several nearest heavy atoms.

In some embodiments, statistical characterization of fragments within the hierarchical library is performed using one or more reference datasets comprising synthesized compounds. Such datasets may include publicly available chemical databases, proprietary datasets, internal experimental collections, or any combination thereof. The choice of dataset or datasets may be adapted based on the intended application, chemical domain, or desired level of conservatism in synthetic accessibility assessment, without departing from the scope of the invention.

In further embodiments, the definition of substituents associated with a fragment may be expanded, modified, or refined to include additional structural detail. For example, substituents may be characterized not only by the immediately attached atom and bond type, but also by descriptions encompassing multiple nearest heavy atoms, extended local environments, or higher-order neighborhood information. Such extensions enable more granular differentiation of attachment contexts and allow the hierarchical library to capture increasingly complex synthetic constraints.

In additional embodiments, the flexibility of the hierarchical representation and statistical framework permits continuous updating, refinement, and reconfiguration of the fragment libraries and associated statistics as new chemical data becomes available. This adaptability allows the invention to be applied across diverse chemical spaces and evolving synthetic practices, while maintaining compatibility with generative chemistry systems, virtual screening pipelines, and other computer-aided molecular design workflows.

In certain embodiments, the hierarchical library of substructures described herein is configurable to include additional layers or to remove existing layers, depending on the desired balance between structural granularity and computational performance. For example, an intermediate layer may be introduced in which substructures are grouped according to generalized substituent atom types, such as any carbon, any nitrogen, any oxygen, or other atomic classes. Such grouping enables efficient pruning of the search space at early stages of substructure matching, thereby maintaining high search performance even as the number of branches in subsequent, more specific layers increases substantially.

In further embodiments, more advanced clustering strategies may be employed within one or more layers of the hierarchy. For instance, atomic primitives associated with multiple substituents may be combined into a single clustered representation, or ring atom primitives from neighboring or structurally similar branches may be aggregated to reflect shared chemical contexts. These clustering techniques allow the hierarchical library to encode higher-order structural relationships while limiting combinatorial growth, thereby improving scalability without sacrificing chemical relevance.

In some embodiments, performance is further enhanced by retaining and reusing intermediate matching results across hierarchical layers. For example, indices of atoms or subgraphs matched at a higher layer of the hierarchy may be stored and propagated to lower layers, eliminating the need to perform a full isomorphic subgraph search for each refinement step. Instead, subsequent searches may be limited to verification of candidate substructures against progressively more specific queries, resulting in significant acceleration of the overall matching process.

In additional embodiments, the algorithm for identifying synthetically irrelevant substructures may be optimized through the use of statistical information associated with intermediate layers of the hierarchy. By aggregating and evaluating occurrence statistics at these intermediate levels, the search process may be terminated prior to reaching the most specific layer when all downstream branches are already associated with unfavorable or penalizing scores. This early-termination strategy further reduces computational overhead while preserving conservative filtering behavior.

Notwithstanding these optional enhancements, the algorithm, even in its baseline implementation, demonstrates performance characteristics sufficient for practical deployment in generative design workflows involving large numbers of candidate small molecules. Accordingly, any of the foregoing technical refinements may be implemented individually or in combination without altering the fundamental principles or scope of the proposed approach, which remains directed to hierarchical, fragment-based representation and filtering of molecular structures based on synthetically relevant substructural features.

In some embodiments, it is also possible to add/remove layers in the hierarchical library. For example, an additional layer could be grouped by the type of substituent atom (any carbon, any nitrogen, etc.). Rational use of this approach will help maintain high search performance even with a significant increase in the number of branches in the final layers. A more complex clustering algorithm could be developed for the layer to combine atomic primitives not only of a single substituent but of several or, for example, to combine ring atom primitives for neighboring similar branches. Significant acceleration can also be achieved by saving the indices of atoms matched at the previous layer so that there is no need to perform an isomorphic subgraph search from scratch but only verify a specific substructure against the refined query. Additionally, the algorithm for searching for irrelevant substructures can be improved. For example, by gathering statistics for intermediate layers, it is possible to stop the search before the final layer if all the lower branches are penalizing. Nevertheless, even in its current state, the algorithm demonstrates excellent performance, sufficient for the tasks of generative design of new small molecules, and any of the abovementioned technical improvements will not lead to substantial change in the matter of the proposed approach.

By creating a hierarchical library that encompasses the substructure space necessary for medicinal chemistry tasks, a fully independent data-driven synthetic accessibility model can be established based on the proposed approach. The outcome of such a model could include, for example, the cumulative statistical contribution of all fragments present in a molecular structure.

In certain embodiments, the approaches and substructure libraries disclosed herein are applied to the analysis of one or more databases of molecular structures to identify, quantify, and characterize trends in substructural motifs. Using the hierarchical representation and associated statistics, the invention enables identification of frequently occurring substructures, as well as rare or underrepresented fragments, within a given chemical dataset. Such analysis may be performed for purposes beyond synthetic accessibility assessment, and the invention is not limited to any particular filtering criterion.

In some embodiments, the hierarchical substructure framework is used to filter or prioritize molecular structures based on criteria other than synthetic feasibility. For example, when applied to a dataset comprising biologically active compounds associated with a defined therapeutic target or biological objective, the invention may be used to identify so-called privileged fragments, enriched motifs, or outlier substructures correlated with desired activity profiles. Conversely, fragments associated with undesired properties may be identified and deprioritized. In this manner, the invention supports structure-property and structure-activity analyses using a unified fragment-centric representation.

In further embodiments, the organized storage of fragment occurrence statistics within the hierarchical library enables longitudinal analysis of chemical datasets over time. By comparing fragment statistics across temporal subsets of a reference database, the invention facilitates tracking of the emergence, evolution, or decline of specific scaffolds, chemotypes, or structural motifs. For example, the system may be used to determine whether new classes of compounds or previously unobserved chemotypes have appeared within a particular dataset during a defined time interval.

In additional embodiments, this capability enables monitoring of the evolution of chemical space within proprietary or public databases, supporting strategic decision-making in medicinal chemistry, library design, and exploratory research. By providing a structured and extensible framework for storing, updating, and querying fragment-level statistics, the invention allows users to observe changes in chemical design trends, assess innovation rates, and identify emerging structural patterns, without departing from the scope of the disclosed hierarchical substructure representation and analysis methodology.

In some embodiments, the proposed approach and resulting substructure libraries can be applied to a database of molecular structures analysis to identify and analyze trending substructures or, conversely, the rarest ones. The aim can be not only to filter based on synthetic feasibility criteria but also on any other criteria. For instance, it may be possible to search for privileged fragments or outlier fragments if the dataset comprises a collection of active compounds targeted at a specific goal. Additionally, this approach is interesting for tracking the emergence of new scaffolds in a reference dataset, as it allows for the organized storage of fragment statistics. Using a developed approach, it is possible to observe an evolution of chemical space, and for example, we can check if any new chemotypes appeared for a period of time in the specific database.

In certain embodiments, knowledge derived from fragment occurrence statistics and the hierarchical organization of fragment relevance is utilized to construct exploitation-oriented generative models. Such models are configured to modify or refine molecular structures by replacing synthetically irrelevant fragments, or fragments deemed irrelevant based on any selected criterion, with structurally and contextually nearest relevant analogs identified within the hierarchical library. This replacement capability enables automated correction and optimization of generated molecules while preserving overall structural intent.

In some embodiments, the hierarchical fragment framework further enables enumeration of nearest scaffold or fragment analogs for the purpose of systematically expanding chemical space. By identifying related and permissible variants of a given scaffold, the invention facilitates controlled exploration of neighboring regions of chemical space, supporting scaffold hopping, analog generation, and diversity expansion in a chemically meaningful manner.

In further embodiments, the accumulated knowledge regarding fragment relevance is applied to guide or constrain other generative modeling approaches. For example, fragment relevance information may be used to establish positive trends by biasing generation toward favorable motifs, or negative trends by penalizing or excluding unfavorable motifs during model training, inference, or post-generation filtering. Such guidance may be applied independently of, or in combination with, synthetic accessibility criteria, and may incorporate biological, physicochemical, or domain-specific relevance metrics.

In additional embodiments, the described use of fragment relevance knowledge enables integration of the hierarchical substructure framework with a wide range of generative architectures, including rule-based, probabilistic, and machine-learning-based models. This integration allows generative systems to leverage curated structural knowledge to improve output quality, reduce unproductive exploration, and align generated compounds with predefined design objectives, without departing from the scope of the invention.

In some embodiments, based on the knowledge of fragment occurrence and the hierarchical structure of this knowledge, exploitation generative models can be constructed, enabling the replacement of synthetically irrelevant fragments (or irrelevant fragments based on any other criterion) with the nearest relevant analogs. The ability to enumerate the nearest analogs of scaffolds for expanding the chemical space is also valuable. On the other hand, the acquired knowledge about fragment relevance can be used to establish both positive and negative trends for any other generative models.

Additionally, the proposed pan-substructural representation of a molecular structure allows for the creation of a hierarchical fingerprint based on the hierarchically organized library of substructures. Such a fingerprint could serve as an alternative to the Morgan fingerprint. The hierarchical structure of the substructures library would allow for considering not only the presence or absence of a fragment but also the hierarchical distance between two fragments and the hierarchical topological distances between fragments as per molecular structure.

In certain embodiments, the pan-substructural representation of a molecular structure disclosed herein enables the construction of a hierarchical molecular fingerprint derived from the hierarchically organized library of substructures. Such a hierarchical fingerprint provides a structured encoding of a molecular structure based on matched substructures across multiple levels of specificity and may serve as an alternative or complement to conventional circular fingerprints, including Morgan fingerprints.

In some embodiments, the hierarchical fingerprint encodes not only the presence or absence of particular substructures, but also relationships between substructures within the hierarchy. For example, similarity between fragments may be quantified based on their hierarchical distance within the substructure library, allowing fragments that share common parent nodes to be treated as more closely related than fragments that diverge at higher hierarchical levels. This enables a graded representation of structural similarity rather than a purely binary encoding.

In further embodiments, the hierarchical fingerprint additionally incorporates topological information reflecting the arrangement of fragments within the molecular graph. Hierarchical topological distances between fragments may be computed based on their relative positions and connectivity in the molecular structure, thereby capturing both local and global structural context. Such combined hierarchical and topological encoding allows the fingerprint to reflect not only which fragments are present, but also how they are organized and related within the molecule.

In additional embodiments, the hierarchical fingerprint may be used in downstream computational tasks including, but not limited to, molecular similarity searching, clustering, classification, regression modeling, structure-activity relationship analysis, and integration with machine-learning models. By leveraging the hierarchical organization of substructures, the fingerprint provides an interpretable and extensible molecular representation that aligns with chemically meaningful fragment relationships, while remaining compatible with existing cheminformatics and computer-aided molecular design workflows.

In some embodiments, a computer-implemented method for analyzing chemical space and modeling synthetic accessibility can include: generating a hierarchical library of SMARTS substructures representing molecular fragments; collecting occurrence statistics for the substructures from reference chemical datasets; identifying synthetically irrelevant substructures based on low occurrence frequency; clustering similar SMARTS strings to optimize the library; and applying a hierarchical filtering algorithm to rapidly identify compounds containing synthetically irrelevant substructures. Accordingly, a synthetic accessibility protocol can then use the hierarchical filtering algorithm to remove synthetically irrelevant substructures from generated molecules. That is, the generated molecules are filtered by synthetic accessibility and molecules with synthetically irrelevant substructures. Therefore, molecules with synthetically relevant substructures are identified with less false positives. These synthetically relevant substructures can then be selected for synthesis and validation.

In some aspects, the method can include generating the hierarchical library by: defining a zero layer comprising a basic aromatic ring structure or other structure; generating subsequent layers by combinatorially replacing atoms in the zero layer structure with atomic primitives from a predefined element alphabet; and removing duplicate substructures through isomorphism checks.

In certain embodiments, the hierarchical library can comprise multiple layers, including: a zero layer representing a basic aromatic ring or other basic chemical moiety; a first layer representing heterocycles with specific numbers and positions of heteroatoms; a second layer representing specific heterocycles; a third layer representing heterocycles with two or three substituents; and a fourth layer representing heterocycles with specific types of substituents in specific positions.

The method can further include defining a substituent alphabet comprising multiple types of substituents, each represented by a specified atom and associated SMARTS notation.

In some implementations, collecting occurrence statistics can involve analyzing reference datasets such as ChEMBL and commercially available compound libraries to determine the frequency of each substructure.

The method may include identifying synthetically irrelevant substructures by applying a frequency threshold, such as flagging substructures that occur fewer than 10 times in the reference datasets.

In certain aspects, clustering similar SMARTS strings can involve automatically combining homogeneous substituents to reduce the number of unique substructures while maintaining chemical relevance.

The hierarchical filtering algorithm can involve: checking for the presence of a basic substructure (e.g. five-membered aromatic heterocycle); if present, checking for substructures from subsequent layers of the library in a hierarchical manner; and terminating the search upon finding a synthetically irrelevant substructure or exhausting all layers.

In some embodiments, the method outputs a Boolean result indicating whether a given molecular structure passes or fails the synthetic accessibility filter, along with atom mappings of any identified irrelevant substructures.

The method can be applied to filter and analyze outputs from generative chemistry models, providing rapid assessment of synthetic accessibility for large numbers of virtual molecular structures.

In another aspect, a system for analyzing chemical space and modeling synthetic accessibility is provided. The system can include one or more processors and memory storing instructions that, when executed, cause the system to perform the methods described herein.

A non-transitory computer-readable medium storing instructions for analyzing chemical space and modeling synthetic accessibility is also provided. The instructions, when executed by one or more processors, cause performance of the methods described herein.

The pan-substructural hierarchical approach described herein provides a novel and efficient method for analyzing chemical space and modeling synthetic accessibility. By leveraging hierarchically organized substructure libraries and statistical analysis of reference datasets, the system enables rapid identification of synthetically challenging molecular structures. This approach has potential applications in various aspects of computational chemistry and drug discovery, particularly in the context of generative chemistry and large-scale virtual screening efforts.

In some embodiments, the reference dataset may comprise molecular structures from the PubChem database, the ChEMBL bioactivity database, the ZINC commercially available compounds database, and various vendor chemical catalogs.

In certain embodiments, the invention provides a computer-implemented approach for analyzing the synthetic accessibility of molecular structures using a hierarchical representation of molecular substructures. The approach is based on the generation of a hierarchical library of molecular fragments, represented using SMARTS strings, which encode possible substructures that may appear within chemical compounds. The hierarchical organization of the library enables efficient traversal and analysis of molecular structures at increasing levels of structural specificity.

In one embodiment, the present invention provides a computer-implemented method for analyzing the synthetic accessibility of molecular structures. The method includes generating a hierarchical library of molecular substructures that represent possible molecular fragments and collecting statistical data describing the occurrence of such substructures within one or more reference chemical datasets. Based on the collected occurrence statistics, substructures exhibiting low frequency of occurrence are identified as synthetically irrelevant. The identified irrelevant substructures are then clustered and optimized to reduce redundancy and improve computational efficiency. A group of molecular structures is subsequently analyzed using a hierarchical filtering algorithm that traverses the hierarchical library to detect the presence of synthetically irrelevant substructures. Molecular structures containing one or more such irrelevant substructures are removed from the group, thereby producing a filtered set of molecular structures that have been screened to exclude structures with reduced likelihood of synthetic feasibility.

In some embodiments, the hierarchical library is constructed by defining an initial or zero layer that represents a general genus of a chemical moiety. This zero layer may correspond, for example, to a basic aromatic five-membered ring, although other cyclic or acyclic structures may also be used. From this zero layer, successive layers of the hierarchy are generated by systematic modification of the underlying SMARTS representations. In one embodiment, a first layer is generated by combinatorially replacing atoms in the zero layer with atomic primitives selected from an element alphabet. A second layer may be generated by replacing atoms with atomic primitives of a different type, thereby increasing structural specificity. Additional layers may be generated by replacing defined symbols with hydrogen atoms or by introducing substituents according to a predefined substituent alphabet. In this manner, progressively more specific substructures are represented across successive layers of the hierarchy.

In further embodiments, the invention includes collecting statistical information regarding the occurrence of the defined substructures within one or more reference chemical datasets comprising synthesized compounds. Such datasets may include publicly available databases, proprietary compound collections, or combinations thereof. By analyzing the frequency with which each substructure appears in the reference datasets, the system derives occurrence statistics that reflect empirical precedent in synthesized chemical space. Frequency thresholds may then be determined and applied to classify substructures as synthetically relevant or synthetically irrelevant, with substructures exhibiting low occurrence frequencies being designated as synthetically irrelevant.

In some embodiments, the library of synthetically irrelevant substructures is further processed through clustering and optimization. This optimization may include combining homogeneous or chemically equivalent substituents within SMARTS representations to reduce redundancy and limit the total number of substructures stored in the library. Such clustering improves computational efficiency while preserving chemically meaningful distinctions between fragment classes.

In certain embodiments, the hierarchical library of irrelevant substructures is used to filter a group of molecular structures. The filtering process may be performed using a hierarchical algorithm that mirrors the structure of the library. For example, an input molecular structure may first be examined for the presence of the defined genus represented by the zero layer, such as a five-membered aromatic heterocycle. If the genus is present, the algorithm proceeds to evaluate whether substructures from the first hierarchical layer are present. When matches are detected, the algorithm continues to progressively deeper layers of the hierarchy. If a substructure corresponding to a terminal or final layer classified as synthetically irrelevant is identified, the molecular structure is rejected. Molecular structures containing one or more irrelevant substructures may be removed from the group to yield a filtered set of molecular structures that are more likely to be synthetically accessible.

In additional embodiments, the invention provides systems implementing the described methods. Such systems may include one or more processors and memory storing instructions that, when executed, cause the system to generate the hierarchical substructure library, collect occurrence statistics, identify synthetically irrelevant substructures, optimize the library, and filter molecular structures using the hierarchical algorithm. In some embodiments, the system comprises functional modules, including a hierarchical library generator, a statistics collector, an irrelevant substructure identifier, a library optimizer, and a hierarchical filter, each configured to perform corresponding operations of the invention.

In further embodiments, the invention is implemented as a non-transitory computer-readable medium storing instructions that, when executed by a processor, cause the processor to perform the synthetic accessibility analysis methods described herein. Such embodiments enable deployment of the invention across a variety of computing environments, including local workstations, servers, and cloud-based platforms.

In some embodiments, the invention further provides for receiving an individual molecular structure as input and producing an output indicative of its synthetic accessibility. The output may comprise a binary acceptance or rejection decision, a synthetic accessibility score, or an annotation identifying the specific substructures deemed synthetically irrelevant. In certain embodiments, the output further includes atom-level mappings corresponding to the matched irrelevant substructures, enabling interpretability and downstream analysis by a chemist or automated system.

In additional embodiments, the hierarchical substructure representation is used to generate a hierarchical molecular fingerprint. Such a fingerprint encodes the presence or absence of substructures at each hierarchical layer and may be used as an alternative or complement to conventional molecular fingerprints. The fingerprint representation supports downstream applications including similarity searching, clustering, classification, regression modeling, and integration with machine-learning systems.

In some embodiments, the invention provides a computer-implemented technique for evaluating the synthetic accessibility of molecular structures. A molecular structure is first received in the form of a SMILES string. The SMILES string is parsed to construct a molecular graph object that represents atoms and bonds within the molecule. The molecular graph object is then analyzed using a hierarchical filtering algorithm that traverses a library of molecular substructures organized into multiple hierarchical layers.

The hierarchical filtering algorithm begins by querying the molecular graph object against a zero layer of the hierarchical library to determine whether a predefined target core structure is present. In certain embodiments, the target core structure is a five-membered aromatic heterocycle. If the target core structure is not present, the filtering process terminates without applying any penalty or restriction to the molecular structure.

When the target core structure is detected, the molecular graph object is sequentially compared against increasingly specific layers of the hierarchical library. Each successive layer represents progressively more detailed structural features of the target core structure, including the number and positions of heteroatoms and the local chemical environments surrounding the core. The filtering algorithm proceeds layer by layer until either no further matches are found or a match is detected in a final layer of the library.

In particular embodiments, the hierarchical library includes a zero layer representing a basic aromatic ring structure, a first layer representing heterocycles having specific numbers and arrangements of heteroatoms, and a second layer representing specific heterocycle types. Additional layers include a third layer that defines heterocycles having two or three substituents at specified positions, and a fourth layer that defines heterocycles having particular substituent types at specific positions. The filtering algorithm terminates when a match is detected in the final layer that has been designated as synthetically irrelevant.

Substructures in the hierarchical library are represented using SMARTS strings. Each SMARTS string encodes both bond primitives and atomic primitives for each substituent position, allowing the substructures to capture detailed local chemical environments. In some embodiments, the SMARTS strings in the final layer are clustered representations that combine multiple homogeneous substituent variations using logical OR operators. This clustering significantly reduces the size of the final-layer library while preserving chemical expressiveness.

Synthetically irrelevant substructures are identified based on statistical occurrence data. Specifically, substructures in the final layer are analyzed against one or more reference datasets of synthesized compounds. Substructures having occurrence frequencies below a predetermined threshold are designated as synthetically irrelevant. In certain embodiments, the threshold is set to approximately ten occurrences, although other thresholds within a defined range may be used.

When a synthetically irrelevant substructure is detected within a molecular graph object, the system may retrieve atom mapping indices corresponding to the matched substructure. These atom mappings can be used to generate a visualization that highlights the synthetically irrelevant region of the molecule. The filtering result is then generated, which in some embodiments is a Boolean output indicating a PASS status when no synthetically irrelevant substructure is detected and a FILTER status when such a substructure is present.

In another embodiment, the invention provides a computer-implemented method for constructing the hierarchical library used for synthetic accessibility evaluation. This process begins by defining a zero-layer core structure as a SMARTS string. An element alphabet is defined that includes atomic primitives for heteroatoms. The first layer of the library is generated by combinatorially replacing generic atom symbols in the zero-layer SMARTS string with atomic primitives from the element alphabet to produce heterocycle-type substructures.

A substituent alphabet is also defined, including bond primitives and atomic primitives corresponding to different substituent types. Subsequent layers are generated by replacing attachment point symbols with hydrogen atoms and substituent placeholders to create position-specific substructures. Further layers enumerate substructures containing two or three substituent positions, followed by a layer in which all substituent types from the substituent alphabet are combinatorially substituted at each substituent position.

Occurrence statistics are collected for each substructure in the final layer by querying one or more reference datasets of synthesized compounds, such as public chemical databases, commercial screening libraries, proprietary compound collections, or combinations thereof. Duplicate substructures are removed using molecular graph isomorphism checks. Substructures that occur below a predetermined frequency threshold are identified as synthetically irrelevant and retained in the hierarchical library with associated statistical metadata.

In some embodiments, the final-layer substructures are further optimized by clustering homogeneous substituent variations. Substructures that differ only in substituent type at one or more positions are grouped together, and their atomic primitives are combined using logical OR operators within a single SMARTS representation. This clustering can reduce the number of final-layer substructures by an order of magnitude while maintaining functional coverage.

In additional embodiments, the hierarchical filtering approach is applied to molecular structures generated by a generative chemistry model. A batch of molecular structures is received, and each structure is independently parsed and filtered using the hierarchical library. Molecular structures that do not contain synthetically irrelevant substructures are assigned a PASS status and included in a filtered output set for synthesis prioritization. Structures containing synthetically irrelevant substructures are assigned a FILTER status. For filtered structures, the system may generate a report identifying the types and frequencies of detected violations.

The hierarchical filtering algorithm may be implemented in a highly efficient manner, allowing processing rates of at least one thousand molecular structures per second on a single processor core. In some embodiments, the filtering results and occurrence statistics are fed back to the generative chemistry model to guide subsequent molecule generation away from synthetically irrelevant motifs.

In further embodiments, the hierarchical library is used to analyze chemical space evolution over time. Multiple temporal snapshots of a reference chemical database are loaded, and occurrence statistics for each substructure are collected at each time point. Newly emerging substructures, trending scaffolds, privileged fragments, and outlier fragments are identified based on changes in occurrence frequency across the temporal snapshots, and corresponding reports are generated.

The hierarchical library may also be used to generate a hierarchical molecular fingerprint for a given molecular structure. The fingerprint encodes the presence or absence of matched substructures, the hierarchical layers in which they appear, and distances between substructures within the hierarchy and within the molecular graph. Such fingerprints may be used for similarity searching, clustering, classification, regression modeling, structure-activity relationship analysis, or integration with machine learning models.

In yet another embodiment, the hierarchical filtering technique is applied during synthetic route planning. Candidate synthetic routes for a target molecule are generated, and intermediate molecular structures are evaluated using the hierarchical filtering algorithm. A synthetic accessibility score is calculated based on the number of intermediates containing synthetically irrelevant substructures, and a preferred route having the lowest score is selected for execution.

In some embodiments, when a target molecule or intermediate is found to contain a synthetically irrelevant substructure, alternative molecular structures are generated by replacing the irrelevant substructure with structurally similar substructures from the hierarchical library that exceed the occurrence frequency threshold. The modified molecule is then used for route generation and synthesis, followed by chemical and/or biological validation.

Once synthetically irrelevant substructures have been identified and molecular structures containing such substructures have been excluded, the remaining molecular structures are designated as synthetically relevant molecules. These molecules are composed exclusively of substructures that meet predefined relevance criteria and are therefore considered suitable for further analysis. The retained set of synthetically relevant molecules forms a refined chemical space that is enriched for structures with a higher likelihood of successful synthesis and practical applicability.

In the next stage of the protocol, synthetically relevant substructures are extracted from the synthetically relevant molecules using the same hierarchical substructure representation employed during filtering. For each retained molecule, substructures are mapped to the hierarchical library to identify the specific fragments and local environments present. Substructures that occur within one or more synthetically relevant molecules are collected and aggregated, thereby forming a set of synthetically relevant substructures supported by empirical occurrence within the filtered chemical space.

The identified synthetically relevant substructures are then characterized according to one or more descriptive criteria. Such characterization may include, but is not limited to, frequency of occurrence within the synthetically relevant molecule set, hierarchical level within the substructure library, chemical diversity of substituent environments, structural complexity, or association with specific scaffolds or molecular classes. Additional annotations may be derived from external data sources, including biological activity data, physicochemical property ranges, or historical synthesis success rates, when such information is available.

Following characterization, the synthetically relevant substructures are ranked according to a defined objective. The ranking criteria may be selected based on the intended downstream application and may include prioritization for synthetic tractability, enrichment in active compounds, suitability for scaffold expansion, or compatibility with generative chemistry workflows. In some implementations, ranking is performed using weighted scoring functions that combine multiple characterization metrics, while in other implementations rule-based or statistical ranking approaches are employed.

Based on the resulting rankings, defined actions may be performed using the synthetically relevant substructures. For example, higher-ranked substructures may be selected as preferred building blocks for de novo molecule generation, scaffold hopping, or focused library design. Alternatively, ranked substructures may be used to bias or constrain generative models, guide medicinal chemistry optimization efforts, or inform decision-making regarding compound prioritization. In this manner, the protocol enables systematic transition from exclusion of synthetically irrelevant chemistry to proactive exploitation of synthetically relevant structural motifs aligned with predefined design goals or desired properties.

Following identification of one or more generated molecules composed exclusively of synthetically relevant substructures, selected candidate molecules are prioritized for chemical synthesis. Selection may be based on predefined criteria, including predicted synthetic feasibility, structural novelty within the filtered chemical space, suitability for downstream biological evaluation, or alignment with a desired functional or therapeutic objective. In some implementations, multiple candidate molecules are selected to provide structural diversity or to enable comparative evaluation of closely related analogs.

For each selected molecule, appropriate reagents, intermediates, and starting materials are obtained or prepared in accordance with the intended synthetic route. Such reagents may include commercially available building blocks, protected intermediates, catalysts, solvents, and reagents required for functional group transformations or coupling reactions. The availability, cost, and handling requirements of these materials may be considered during selection and procurement to ensure practical execution of the synthesis. Where applicable, alternative reagents or routes may be identified to mitigate supply or scalability constraints.

The selected molecules are then synthesized using suitable chemical synthesis techniques consistent with their structural features. The synthesis may involve one or more steps, including bond-forming reactions, functional group interconversions, protection and deprotection steps, cyclization reactions, or purification processes. Upon completion of synthesis, crude reaction products are subjected to purification using appropriate methods, such as crystallization, liquid-liquid extraction, flash chromatography, preparative high-performance liquid chromatography (HPLC), or other separation techniques, to obtain isolated compounds of sufficient purity for characterization and testing.

Following purification, the synthesized molecules are chemically characterized to confirm their identity, structure, and purity. Chemical characterization may include nuclear magnetic resonance (NMR) spectroscopy, such as one-dimensional and two-dimensional 1H, 13C, COSY, HSQC, and HMBC experiments, to verify structural connectivity and stereochemical features. Additional characterization techniques may include mass spectrometry (e.g., LC-MS, HRMS) to confirm molecular weight and composition, infrared (IR) spectroscopy to assess functional groups, and elemental analysis to verify empirical composition. Chromatographic methods, such as analytical HPLC, gas chromatography (GC), or thin-layer chromatography (TLC), may be used to assess purity, retention behavior, and batch consistency. Where applicable, solid-state characterization methods, including melting point determination, X-ray crystallography, or polymorph analysis, may also be employed.

Once chemical identity and purity are confirmed, the synthesized molecules are subjected to biological characterization to evaluate whether they exhibit the defined function or property of interest. Biological testing may include in vitro biochemical assays, cell-based assays, or other relevant experimental systems depending on the intended application. For example, immunoassays such as enzyme-linked immunosorbent assays (ELISA) may be used to measure binding interactions or modulation of specific proteins, while Western blotting may be employed to assess effects on protein expression, modification, or signaling pathways. Additional biological characterization methods may include reporter gene assays, enzyme inhibition or activation assays, receptor binding assays, fluorescence- or luminescence-based readouts, or high-content imaging assays.

In some embodiments, further biological evaluation may include cell viability or cytotoxicity assays, phenotypic screening, or functional assays assessing cellular responses such as proliferation, differentiation, or apoptosis. Where relevant, biophysical techniques such as surface plasmon resonance (SPR), isothermal titration calorimetry (ITC), or thermal shift assays may be used to characterize binding affinity and mechanism of action. The results of these biological assays are compared against predefined benchmarks or control compounds to determine whether the synthesized molecules exhibit the desired activity profile or functional property.

Collectively, this protocol establishes a closed-loop workflow in which molecules generated using synthetically relevant substructures are synthesized, chemically validated, and biologically evaluated. The resulting data may be used to confirm the practical utility of the generative design process, refine selection criteria, and inform subsequent rounds of molecule generation, optimization, or experimental validation.

In certain embodiments, once a synthesized molecule has been chemically and biologically characterized and confirmed to exhibit a designed property, the molecule is employed for its intended use. Such use may occur in a research, diagnostic, therapeutic, industrial, or commercial context, depending on the nature of the defined property and the application for which the molecule was generated. The invention contemplates utilization of the synthesized molecule in any environment in which its verified functional characteristics provide a practical benefit.

In some embodiments, the synthesized molecule exhibits a biological activity, such as binding to a target protein, modulating enzymatic activity, altering a signaling pathway, or influencing gene expression. In such cases, the molecule may be used as a biochemical probe, screening reagent, or reference compound in research assays. For example, a molecule identified as an inhibitor or activator of a specific protein may be used in in vitro or cell-based assays to study biological mechanisms, validate targets, or assess pathway modulation. In other embodiments, the molecule may serve as a lead compound or optimized candidate for further development in therapeutic discovery programs.

In further embodiments, the synthesized molecule exhibits diagnostic or analytical properties. For example, the molecule may possess selective binding affinity for a biomarker, antigen, or molecular target, enabling its use in diagnostic assays, imaging applications, or detection platforms. Such molecules may be incorporated into assay kits, labeled detection reagents, or analytical workflows for identifying, quantifying, or monitoring biological or chemical entities. In some implementations, the molecule may be conjugated to a reporter, surface, or carrier to facilitate detection or signal generation.

In additional embodiments, the synthesized molecule exhibits physicochemical properties that make it suitable for use in materials science, chemical manufacturing, or industrial processes. For instance, the molecule may function as a catalyst, ligand, stabilizer, additive, or intermediate in a chemical reaction or formulation. In such embodiments, the molecule is used to improve reaction efficiency, selectivity, stability, or performance of a chemical system. Alternatively, the molecule may serve as a building block for further chemical modification or assembly into more complex structures.

In some embodiments, the synthesized molecule exhibits favorable absorption, distribution, metabolism, excretion, or stability characteristics and is therefore used in preclinical or applied testing environments. For example, the molecule may be administered in experimental models to evaluate pharmacological behavior, toxicity, or efficacy. In other embodiments, the molecule may be incorporated into formulation studies, delivery systems, or combination products designed to achieve a specific functional outcome.

In all such embodiments, the use of the synthesized molecule is enabled by the prior identification of synthetically relevant substructures and the verification that the molecule possesses the designed property or function. The ability to generate, synthesize, validate, and ultimately deploy molecules according to predefined objectives demonstrates the practical applicability of the disclosed approach across a wide range of chemical, biological, and industrial use cases. The invention is not limited to any particular property or application, and the synthesized molecules may be used for any purpose consistent with their characterized functionality.

### Embodiments

In some embodiments, a computer-implemented method for evaluating synthetic accessibility and/or improving synthetic accessibility analysis of molecular structures can include receiving a molecular structure represented as a SMILES string. The method may include parsing the SMILES string to generate a molecular graph object. The method may include applying a hierarchical filtering algorithm to the molecular graph object. The hierarchical filtering algorithm may include querying the molecular graph object against a zero layer of a hierarchical library to determine presence of a target core structure. When the target core structure is present, the hierarchical filtering algorithm may include sequentially querying the molecular graph object against successive layers of the hierarchical library. Each successive layer may represent progressively more specific structural features of the target core structure with local chemical environment. The hierarchical filtering algorithm may include terminating upon detecting a match with a synthetically irrelevant substructure from a final layer of the hierarchical library. The method may include generating a filtering result indicating whether the molecular structure contains a synthetically irrelevant substructure based on the hierarchical filtering algorithm.

In some embodiments, the target core structure comprises a five-membered aromatic heterocycle or other chemical moiety.

In some embodiments, the hierarchical library may include the zero layer representing a basic aromatic ring structure. The hierarchical library may include a first layer representing heterocycles with specific numbers and positions of heteroatoms. The hierarchical library may include a second layer representing specific heterocycle types. The hierarchical library may include a third layer representing heterocycles with two or three substituents at defined positions. The hierarchical library may include a fourth layer representing heterocycles with specific types of substituents at specific positions.

In some embodiments, the synthetically irrelevant substructure is identified from the fourth layer based on occurrence frequency in a reference dataset of synthesized compounds being below a predetermined threshold.

In some embodiments, the predetermined threshold is 10 occurrences in the reference dataset.

In some embodiments, each substructure in the hierarchical library is represented as a SMARTS string that encodes both a bond primitive and an atomic primitive for each substituent position.

In some embodiments, the SMARTS strings in the final layer comprise clustered representations combining multiple homogeneous substituent variations using logical OR operators.

In some embodiments, wherein the hierarchical filtering algorithm terminates without applying a penalty when: the target core structure is absent from the molecular graph object; or no match is found in any layer of the hierarchical library.

In some embodiments, the method further comprises: when a synthetically irrelevant substructure is detected, retrieving atom mapping indices corresponding to the synthetically irrelevant substructure within the molecular graph object; and generating a visualization highlighting the synthetically irrelevant substructure based on the atom mapping indices.

In some embodiments, the filtering result comprises a Boolean value indicating PASS when no synthetically irrelevant substructure is detected and FILTER when a synthetically irrelevant substructure is detected.

In some embodiments, a computer-implemented method for constructing a hierarchical library of molecular substructures for synthetic accessibility evaluation is provided. The method may include defining a zero layer comprising a core structure represented as a SMARTS string. The method may include defining an element alphabet comprising atomic primitives for heteroatoms. The method may include generating a first layer by combinatorially replacing generic atom symbols in the zero layer SMARTS string with atomic primitives from the element alphabet to produce heterocycle type substructures. The method may include defining a substituent alphabet comprising bond primitives and atomic primitives for substituent types. The method may include generating a second layer by replacing attachment point symbols in the first layer substructures with hydrogen atoms and substituent placeholders to produce position-specific substructures. The method may include generating a third layer by enumerating substructures having two or three substituent positions. The method may include generating a fourth layer by combinatorially substituting all substituent types from the substituent alphabet at each substituent position in the third layer substructures. The method may include collecting occurrence statistics for each substructure in the fourth layer by querying a reference dataset of synthesized compounds. The method may include identifying synthetically irrelevant substructures as those having occurrence frequency below a predetermined threshold. The method may include storing the hierarchical library comprising the zero layer, first layer, second layer, third layer, and fourth layer with associated occurrence statistics.

In some embodiments, the method further comprises removing duplicate substructures by performing isomorphism checks between molecular graph representations of the substructures.

In some embodiments, the method may further include clustering homogeneous substituent variations in the fourth layer. The clustering may include identifying groups of substructures differing only in substituent type at one or more positions. The clustering may include combining atomic primitives for the varying substituents using logical OR operators. The clustering may include replacing the groups of substructures with single clustered SMARTS strings.

In some embodiments, the clustering reduces the number of substructures in the fourth layer from approximately 46,000 to approximately 7,000.

In some embodiments, the reference dataset comprises at least one of: a ChEMBL database comprising bioactive molecules; an Enamine screening compound library; a proprietary pharmaceutical compound collection; or a combination thereof.

In some embodiments, the substituent alphabet comprises at least 18 substituent types, each substituent type defined by: a symbol identifier; a bond primitive specifying bond type and ring membership; and an atomic primitive specifying atom type and local chemical environment.

In some embodiments, the element alphabet is limited to heterocycles containing up to three heteroatoms. In some embodiments, the predetermined threshold is selected from a range of 5 to 20 occurrences.

In some embodiments, a computer-implemented method for filtering molecular structures generated by a generative chemistry model is provided. The method may include receiving a batch of molecular structures from a generative chemistry model. Each molecular structure may be represented as a SMILES string. For each molecular structure in the batch, the method may include parsing the SMILES string to generate a molecular graph object. The method may include applying a hierarchical filtering algorithm to determine whether the molecular graph object contains a synthetically irrelevant substructure by traversing a hierarchical library of SMARTS-encoded substructures. The method may include assigning a PASS status when no synthetically irrelevant substructure is detected. The method may include assigning a FILTER status when a synthetically irrelevant substructure is detected. The method may include generating a filtered set comprising molecular structures having PASS status. The method may include outputting the filtered set for synthesis prioritization.

In some embodiments, the method further comprises: for each molecular structure having FILTER status, identifying the synthetically irrelevant substructure and corresponding atom mapping indices; and generating a report comprising statistics on violation types and frequencies.

In some embodiments, the hierarchical filtering algorithm achieves a processing rate of at least 1,000 molecular structures per second on a single CPU.

In some embodiments, the method further comprises: providing feedback to the generative chemistry model comprising:—a list of synthetically irrelevant substructures to avoid; and—occurrence statistics from a reference dataset for each synthetically irrelevant substructure.

In some embodiments, a computer-implemented method for analyzing chemical space evolution is provided. The method may include loading multiple temporal snapshots of a reference chemical database. The method may include applying a hierarchical library of molecular substructures to each temporal snapshot to collect occurrence statistics for each substructure at each time point. The method may include identifying newly emerged substructures having zero occurrences in earlier snapshots and non-zero occurrences in later snapshots. The method may include calculating growth rates for existing substructures across temporal snapshots. The method may include identifying trending scaffolds based on the growth rates. The method may include generating a report comprising chemical space evolution metrics and trending scaffold identifications.

In some embodiments, the method includes identifying privileged fragments as substructures having consistently high occurrence across all temporal snapshots.

In some embodiments, the method includes identifying outlier fragments as substructures having occurrence frequency below a predetermined threshold across all temporal snapshots.

In some embodiments, a computer-implemented method for generating a hierarchical molecular fingerprint is provided. The method may include receiving a molecular structure represented as a SMILES string. The method may include parsing the SMILES string to generate a molecular graph object. The method may include matching the molecular graph object against a hierarchical library of substructures organized in multiple layers. For each matched substructure, the method may include recording presence of the substructure. The method may include recording layer position of the substructure within the hierarchical library. The method may include recording hierarchical distance to other matched substructures. The method may include encoding the recorded information as a hierarchical fingerprint comprising a binary vector indicating presence or absence of substructures and a distance matrix encoding hierarchical relationships between matched substructures. The method may include outputting the hierarchical fingerprint for use in molecular similarity analysis.

In some embodiments, the method can include incorporating topological distances between matched substructures within the molecular graph object into the hierarchical fingerprint.

In some embodiments, the hierarchical fingerprint is used for at least one of: molecular similarity searching; clustering of molecular structures; classification of molecular structures; regression modeling; structure-activity relationship analysis; or integration with machine learning models.

In some embodiments, a computer-implemented method for selecting a synthetic route for a target molecule is provided. The method may include receiving a target molecule represented as a SMILES string. The method may include generating a plurality of candidate synthetic routes for the target molecule. For each candidate synthetic route, the method may include identifying intermediate molecular structures. The method may include applying a hierarchical filtering algorithm to each intermediate molecular structure to determine presence of synthetically irrelevant substructures. The method may include calculating a synthetic accessibility score based on number of intermediate molecular structures containing synthetically irrelevant substructures. The method may include selecting a preferred synthetic route having a lowest synthetic accessibility score. The method may include outputting the preferred synthetic route for synthesis execution.

In some embodiments, the method includes synthesizing the target molecule according to the preferred synthetic route.

In some embodiments, the method includes validating the synthesized target molecule by at least one of:—chemical validation comprising spectroscopic analysis; or—biological validation comprising activity assay.

In some embodiments, a computer-implemented method for synthesizing a target molecule is provided. The method may include receiving a target molecule represented as a SMILES string. The method may include applying a hierarchical filtering algorithm to the target molecule to determine whether the target molecule contains a synthetically irrelevant substructure. When the target molecule contains a synthetically irrelevant substructure, the method may include identifying the synthetically irrelevant substructure. The method may include generating alternative molecular structures by replacing the synthetically irrelevant substructure with structurally similar substructures from a hierarchical library that are not synthetically irrelevant. The method may include selecting a modified target molecule from the alternative molecular structures. When the target molecule does not contain a synthetically irrelevant substructure or after selecting the modified target molecule, the method may include generating a synthetic route for the target molecule or modified target molecule. The method may include executing the synthetic route to synthesize the target molecule or modified target molecule. The method may include validating the synthesized molecule.

In some embodiments, the validating of the synthesized molecule comprises performing chemical validation by at least one of: nuclear magnetic resonance spectroscopy; mass spectrometry; high-performance liquid chromatography; or infrared spectroscopy.

In some embodiments, validating the synthesized molecule comprises performing biological validation by at least one of: binding affinity assay; enzyme inhibition assay; cell-based activity assay; or in vivo efficacy study.

In some embodiments, generating alternative molecular structures comprises: identifying a parent node in the hierarchical library corresponding to the synthetically irrelevant substructure; identifying sibling nodes of the parent node representing structurally similar substructures; filtering the sibling nodes to retain only substructures having occurrence frequency above a predetermined threshold; and generating the alternative molecular structures by substituting the synthetically irrelevant substructure with each retained sibling substructure.

In some embodiments, a system for evaluating or improving synthetic accessibility and/or improving synthetic accessibility analysis of molecular structures is provided. The system may include one or more processors. The system may include memory storing instructions that, when executed by the one or more processors, cause the system to perform functions. The functions can include receiving a molecular structure represented as a SMILES string. The functions can include parsing the SMILES string to generate a molecular graph object. The functions can include applying a hierarchical filtering algorithm to the molecular graph object by traversing a hierarchical library of SMARTS-encoded substructures organized in multiple layers. The functions can include determining whether the molecular graph object contains a synthetically irrelevant substructure based on the hierarchical filtering algorithm. The functions can include generating a filtering result indicating synthetic accessibility of the molecular structure.

In some embodiments, the hierarchical library is stored in the memory and comprises: a zero layer representing a basic aromatic ring structure or other chemical moiety; a first layer representing heterocycles with specific heteroatom arrangements; a second layer representing specific heterocycle types; a third layer representing heterocycles with defined substituent positions; and a fourth layer representing heterocycles with specific substituent types, wherein substructures in the fourth layer having occurrence frequency below a predetermined threshold in a reference dataset are designated as synthetically irrelevant.

In some embodiments, the instructions further cause the system to: process the molecular structure in parallel with other molecular structures using multiple processor cores; and achieve a processing rate of at least 1,000 molecular structures per second.

In some embodiments, a non-transitory computer-readable medium storing instructions that, when executed by one or more processors, cause performance of a method is provided. The method can include receiving a molecular structure represented as a SMILES string. The method can include applying a hierarchical filtering algorithm to the molecular structure to determine presence of a synthetically irrelevant substructure. The applying may include checking for presence of a target core structure. When the target core structure is present, the applying may include sequentially checking for presence of progressively more specific structural features across multiple layers of a hierarchical library. The applying may include terminating upon detecting a match with a synthetically irrelevant substructure from a final layer of the hierarchical library. The method can include generating a filtering result indicating whether the molecular structure is synthetically accessible. The method can include outputting the filtering result.

In some embodiments, the instructions further cause the implementation that when the molecular structure is determined to be synthetically inaccessible, the computer generates synthesis instructions for an alternative molecular structure lacking the synthetically irrelevant substructure.

In some embodiments, a computer-implemented method for analyzing synthetic accessibility and/or improving synthetic accessibility analysis of molecular structures is provided. The method can include generating a hierarchical library of molecule substructures representing possible molecular fragments. The method can include collecting statistics on substructure occurrences in reference chemical datasets. The method can include identifying synthetically irrelevant substructures based on the occurrence statistics, wherein low occurrence statistics indicate the synthetically irrelevant substructures. The method can include clustering and optimizing the library of irrelevant substructures. The method can include filtering a group of molecular structures using a hierarchical algorithm to identify those containing irrelevant substructures. The method can include removing the molecular structures containing irrelevant substructures from the group of molecular structures to obtain a filtered set of molecular structures that are filtered by removing molecular structures having synthetically irrelevant substructures.

In some embodiments, generating the hierarchical library comprises: defining a zero layer comprising a defined genus of a chemical moiety, optionally as a basic aromatic five-membered ring or other chemical moiety structure; generating a first layer by combinatorically replacing atoms in the zero layer with atomic primitives from an element alphabet; generating a second layer by replacing atoms with atomic primitives of a different type; generating a third layer by replacing a defined symbol with a hydrogen atom in the second-layer strings; and generating at least one subsequent layer by replacing substituents according to a substituent alphabet. In some embodiments, collecting statistics comprises: analyzing substructure occurrences in reference datasets of synthesized compounds; and determining frequency thresholds for classifying substructures as synthetically relevant or irrelevant.

In some embodiments, clustering and optimizing the library comprises: combining homogeneous substituents in SMARTS strings to reduce the number of substructures.

In some embodiments, the hierarchical filtering algorithm comprises: checking for the presence of the defined genus of a chemical moiety, optionally a five-membered aromatic heterocycle or other structure; if present, checking for substructures from the first layer of the library; proceeding to subsequent layers if matches are found; and terminating and rejecting the structure if a match is found in the final layer.

In some embodiments, a system for analyzing synthetic accessibility and/or improving synthetic accessibility analysis of molecular structures is provided. The system can include a processor. The system can include a memory storing instructions that, when executed by the processor, cause the system to perform a computing method. The computing method can include generating a hierarchical library of SMARTS substructures representing possible molecular fragments. The computing method can include collecting statistics on substructure occurrences in reference chemical datasets. The computing method can include identifying synthetically irrelevant substructures based on the occurrence statistics. The computing method can include clustering and optimizing the library of irrelevant substructures. The computing method can include filtering molecular structures using a hierarchical algorithm to identify those containing irrelevant substructures.

In some embodiments, the instructions to generate the hierarchical library comprise instructions to: define a zero layer comprising a basic aromatic five-membered ring or other chemical moiety; generate a first layer by combinatorically replacing atoms in the zero layer with atomic primitives from an element alphabet; generate a second layer by replacing atoms with atomic primitives of a different type; and generate subsequent layers by replacing substituents according to a substituent alphabet.

In some embodiments, the instructions to collect statistics comprise instructions to: analyze substructure occurrences in reference datasets of synthesized compounds; and
determine frequency thresholds for classifying substructures as synthetically relevant or irrelevant.

In some embodiments, the instructions to cluster and optimize the library comprise instructions to combine homogeneous substituents in SMARTS strings to reduce the number of substructures.

In some embodiments, the hierarchical filtering algorithm comprises: checking for the presence of a five-membered aromatic heterocycle or other chemical moiety; if present, checking for substructures from the first layer of the library; proceeding to subsequent layers if matches are found; and terminating and rejecting the structure if a match is found in the final layer. If not present, terminating.

In some embodiments, a non-transitory computer-readable medium is provided for storing instructions that, when executed by a processor, cause the processor to perform a method for analyzing synthetic accessibility and/or improving synthetic accessibility analysis of molecular structures. In some aspects, the method can include generating a hierarchical library of SMARTS substructures representing possible molecular fragments. The method can include collecting statistics on substructure occurrences in reference chemical datasets. The method can include identifying synthetically irrelevant substructures based on the occurrence statistics. The method can include clustering and optimizing the library of irrelevant substructures. The method can include filtering molecular structures using a hierarchical algorithm to identify those containing irrelevant substructures.

In some embodiments, generating the hierarchical library comprises: defining a zero layer comprising a basic aromatic five-membered ring or other chemical moiety; generating a first layer by combinatorically replacing atoms in the zero layer with atomic primitives from an element alphabet; generating a second layer by replacing atoms with atomic primitives of a different type; and generating subsequent layers by replacing substituents according to a substituent alphabet.

In some embodiments, collecting statistics comprises: analyzing substructure occurrences in reference datasets of synthesized compounds; and determining frequency thresholds for classifying substructures as synthetically relevant or irrelevant.

In some embodiments, the hierarchical filtering algorithm comprises: checking for the presence of a five-membered aromatic heterocycle or other chemical moiety; if present, checking for substructures from the first layer of the library; proceeding to subsequent layers if matches are found; and terminating and rejecting the structure if a match is found in the final layer.

In some embodiments, a system for analyzing synthetic accessibility and/or improving synthetic accessibility analysis of molecular structures is provided. The system may include a hierarchical library generator configured to generate a hierarchical library of SMARTS substructures representing possible molecular fragments. The system may include a statistics collector configured to collect occurrence statistics for the substructures in reference chemical datasets. The system may include an irrelevant substructure identifier configured to identify synthetically irrelevant substructures based on the occurrence statistics. The system may include a library optimizer configured to cluster and optimize the library of irrelevant substructures. The system may include a hierarchical filter configured to filter molecular structures using a hierarchical algorithm to identify those containing irrelevant substructures.

In some embodiments, the hierarchical library generator is configured to generate the hierarchical library by: defining a zero layer comprising a basic aromatic five-membered ring or other chemical moiety; generating a first layer by combinatorically replacing symbols in the zero layer with atomic primitives from an element alphabet; generating a second layer by replacing symbols in the first layer with atomic primitives; and generating subsequent layers by replacing symbols with substituents.

In some embodiments, the statistics collector is configured to analyze substructure occurrences in the ChEMBL database and the Enamine compound collection.

In some embodiments, the library optimizer is configured to combine homogeneous substituents in SMARTS strings to reduce the number of penalizing substructures.

In some embodiments, the hierarchical filter is configured to: check for the presence of a five-membered aromatic heterocycle or another chemical moiety in an input molecular structure; if present, check for substructures from the first layer of the hierarchical library; and continue checking subsequent layers until finding a match or exhausting all layers.

In some embodiments, a non-transitory computer-readable medium storing instructions that, when executed by a processor, cause the processor to perform a method for analyzing synthetic accessibility and/or improving synthetic accessibility analysis of molecular structures is provided. The method can include generating a hierarchical library of SMARTS substructures representing possible molecular fragments. The method can include collecting occurrence statistics for the substructures in reference chemical datasets. The method can include identifying synthetically irrelevant substructures based on the occurrence statistics. The method can include clustering and optimizing the library of irrelevant substructures. The method can include filtering molecular structures using a hierarchical algorithm to identify those containing irrelevant substructures.

In some embodiments, generating the hierarchical library comprises: defining a zero layer comprising a basic aromatic five-membered ring; generating a first layer by combinatorically replacing symbols in the zero layer with atomic primitives from an element alphabet; generating a second layer by replacing symbols in the first layer with atomic primitives; and generating subsequent layers by replacing symbols with substituents.

In some embodiments, collecting occurrence statistics comprises analyzing substructure occurrences in the ChEMBL database and the Enamine compound collection.

In some embodiments, clustering and optimizing the library comprises combining homogeneous substituents in SMARTS strings to reduce the number of penalizing substructures.

In some embodiments, filtering molecular structures comprises: checking for the presence of a five-membered aromatic heterocycle or another chemical moiety in an input molecular structure; if present, checking for substructures from the first layer of the hierarchical library; and continuing to check subsequent layers until finding a match or exhausting all layers.

In some embodiments, a computer-implemented method for analyzing synthetic accessibility and/or improving synthetic accessibility analysis of molecular structures is provided. The method comprises: generating a hierarchical library of SMARTS substructures representing possible molecular fragments; collecting statistics on substructure occurrences in reference chemical datasets; identifying synthetically irrelevant substructures based on the occurrence statistics; clustering and optimizing the library of irrelevant substructures; receiving an input molecular structure; filtering the input molecular structure using a hierarchical algorithm to identify irrelevant substructures; and outputting a synthetic accessibility assessment for the input molecular structure based on the filtering.

In some embodiments, generating the hierarchical library comprises: defining a zero layer representing a basic aromatic five-membered ring or another chemical moiety; generating a first layer by combinatorically replacing symbols in the zero layer with atomic primitives from an element alphabet; generating a second layer by replacing symbols in the first layer with additional atomic primitives; and generating third and fourth layers by replacing symbols with substituents.

In some embodiments, identifying synthetically irrelevant substructures comprises selecting substructures occurring fewer than 10 times in the reference datasets.

In some embodiments, clustering and optimizing comprises automatically combining homogeneous substituents in SMARTS strings.

In some embodiments, the hierarchical filtering algorithm comprises: checking for presence of a five-membered aromatic heterocycle or another chemical moiety; if present, checking for substructures from successive layers of the library; and terminating upon finding a substructure from the final layer or exhausting all layers.

In some embodiments, the method includes outputting atom mappings for any identified irrelevant substructures.

In some embodiments, a method for generating a hierarchical fingerprint of a molecular structure is provided. The method can include generating a hierarchical library of SMARTS substructures representing possible molecular fragments. The method can include organizing the substructures into hierarchical layers based on structural complexity. The method can include analyzing the molecular structure to identify the presence of substructures from each hierarchical layer. The method can include generating a fingerprint encoding the presence or absence of substructures at each hierarchical level.

In some embodiments, a system for generating a hierarchical fingerprint of a molecular structure is provided. The system can include one or more processors. The system can include memory storing instructions that, when executed by the one or more processors, cause the system to perform functions. The functions can include generating a hierarchical library of SMARTS substructures representing possible molecular fragments. The functions can include organizing the substructures into hierarchical layers based on structural complexity. The functions can include analyzing the molecular structure to identify the presence of substructures from each hierarchical layer. The functions can include generating a fingerprint encoding the presence or absence of substructures at each hierarchical level.

Finally, the invention may be implemented as a system including one or more processors and memory storing instructions that perform the described methods, or as a non-transitory computer-readable medium storing instructions that cause a processor to perform the hierarchical filtering, library construction, and synthetic accessibility evaluation techniques described above.

Collectively, these embodiments describe a flexible and extensible framework for representing, analyzing, and filtering molecular structures based on hierarchical substructural knowledge derived from real chemical space. While specific embodiments may reference five-membered aromatic heterocycles, particular datasets, or specific threshold values, the invention is not limited thereto, and the described principles may be applied broadly to other classes of molecular fragments, datasets, and analytical objectives without departing from the scope of the invention.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In one embodiment, the present methods can include aspects performed on a computing system. As such, the computing system can include a memory device that has the computer-executable instructions for performing the methods. The computer-executable instructions can be part of a computer program product that includes one or more protocols or algorithms for performing any of the methods of any of the claims.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor.

There are various vehicles by which processes and/or systems and/or other technologies described herein can be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

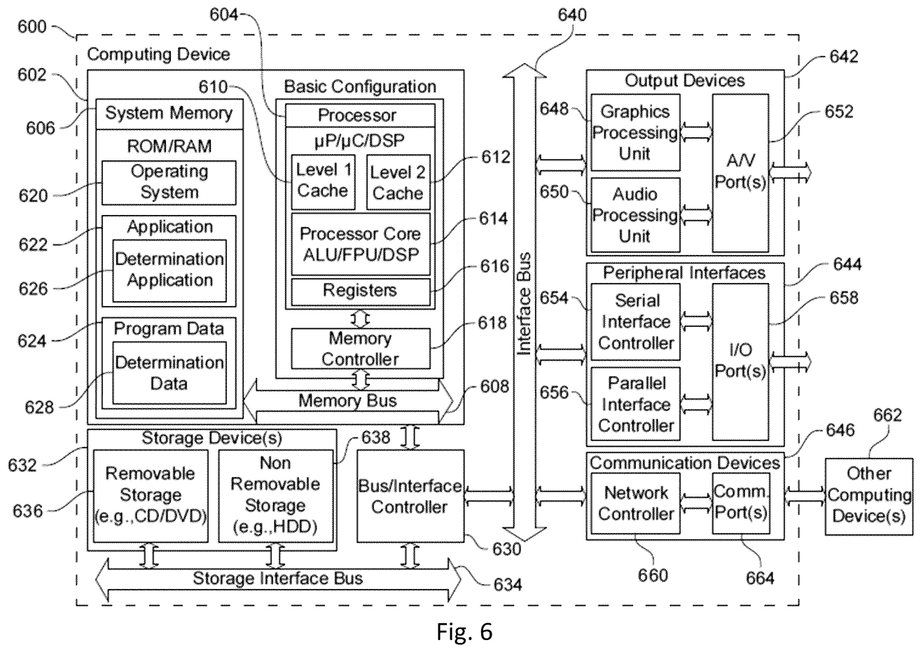

FIG. 6 shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (P), a microcontroller (C), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 can obtain data (e.g., determination data 628), such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In some embodiments, a computer program product can include a non-transient, tangible memory device having computer-executable instructions that when executed by a processor, cause performance of a method that can include: providing a dataset having object data for an object and condition data for a condition; processing the object data of the dataset to obtain latent object data and latent object-condition data with an object encoder; processing the condition data of the dataset to obtain latent condition data and latent condition-object data with a condition encoder; processing the latent object data and the latent object-condition data to obtain generated object data with an object decoder; processing the latent condition data and latent condition-object data to obtain generated condition data with a condition decoder; comparing the latent object-condition data to the latent-condition data to determine a difference; processing the latent object data and latent condition data and one of the latent object-condition data or latent condition-object data with a discriminator to obtain a discriminator value; selecting a selected object from the generated object data based on the generated object data, generated condition data, and the difference between the latent object-condition data and latent condition-object data; and providing the selected object in a report with a recommendation for validation of a physical form of the object. The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

In some embodiments, a computer program product can include a non-transient, tangible memory device having computer-executable instructions that when executed by a processor, cause performance of a method described herein.

The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

### Definitions

A split is a set of fragments and building blocks resulting from an independent branch of a retrosynthetic tree. One molecule can be splitted into multiple splits if many robust reactions can be applied to the starting molecule itself and to the intermediate fragments until the set of available reactions is exhausted.

A representative chemical space is a fraction of the chemical space that provides statistical clarity and the shape of normal distribution. The use of representative chemical space is extremely important since this assumption allows us not to explore the whole almost infinite chemical space. The best fit for that role are ready-to-use compound aggregators like open-sourced PubChem, ZINC and ChEMBL or vendor screening stocks like those from ChemDiv Inc. and Enamine Inc. or commercial datasets.

A robust reaction is an organic reaction or a group of similar reactions, or a general synthetic method which is widely used in the practice of organic synthesis of small drug molecules.

A “synthon-like fragment” (later fragment) is a synthon-like part of a molecule that is produced after cheminformatics decomposition (virtual reaction) by a robust reaction. A fragment is not a real chemical structure, but possibly can be converted into a building block by cheminformatics transformation to become a real compound structure. The resulting building block can be checked for availability in building blocks vendor datasets.

A building block is a real (valid) chemical structure that can be found in the vendor datasets. Building blocks can be produced directly from some cheminformatics decompositions (virtual reactions) or by conversion from fragments (conversion rules). The resulting building blocks can be checked for availability in vendor datasets such as ChemDiv and Enamine. The availability of building blocks in the vendor datasets is considered as a factor of higher synthetic accessibility.

All references recited herein are incorporated herein by specific reference in their entirety.
1. Warr W. A. Combinatorial Chemistry and Molecular Diversity. An Overview//J. Chem. Inf. Comput. Sci. American Chemical Society, 1997. Vol. 37, No 1. P. 134-140.
2. Bohacek R. S., McMartin C., Guida W. C. The art and practice of structure-based drug design: a molecular modeling perspective/Med. Res. Rev. Wiley Online Library, 1996. Vol. 16, No 1. P. 3-50.
3. Ertl P. Cheminformatics analysis of organic substituents: identification of the most common substituents, calculation of substituent properties, and automatic identification of drug-like bioisosteric groups//J. Chem. Inf. Comput. Sci. ACS Publications, 2003. Vol. 43, Ns 2. P. 374-380.
4. Polishchuk P. G., Madzhidov T. I., Varnek A. Estimation of the size of drug-like chemical space based on GDB-17 data//J. Comput. Aided Mol. Des. Springer, 2013. Vol. 27, N 8. P. 675-679.
5. Gao W., Coley C. W. The Synthesizability of Molecules Proposed by Generative Models//J. Chem. Inf. Model. ACS Publications, 2020. Vol. 60, No 12. P. 5714-5723.
6. Bilodeau C. et al. Generative models for molecular discovery: Recent advances and challenges//Wiley Interdiscip. Rev. Comput. Mol. Sci. Wiley, 2022. Vol. 12, No 5.
7. Vanhaelen Q., Lin Y.-C., Zhavoronkov A. The Advent of Generative Chemistry//ACS Med. Chem. Lett. ACS Publications, 2020. Vol. 11, No 8. P. 1496-1505.
8. Bian Y., Xie X.-Q. Generative chemistry: drug discovery with deep learning generative models//J. Mol. Model. Springer, 2021. Vol. 27, No 3. P. 71.
9. Ivanenkov Y. et al. The Hitchhiker's Guide to Deep Learning Driven Generative Chemistry//ACS Med. Chem. Lett. ACS Publications, 2023. Vol. 14, No 7. P. 901-915.
10. Goldman B. et al. Defining Levels of Automated Chemical Design//J. Med. Chem. ACS Publications, 2022. Vol. 65, No 10. P. 7073-7087.
11. Anstine D. M., Isayev O. Generative Models as an Emerging Paradigm in the Chemical Sciences//J. Am. Chem. Soc. ACS Publications, 2023. Vol. 145, NX 16. P. 8736-8750.
12. Du Y. et al. Machine learning-aided generative molecular design//Nat. Mach. Intell. Springer Science and Business Media LLC, 2024. Vol. 6, No 6. P. 589-604.
13. Lipinski C. A. et al. Experimental and computational approaches to estimate solubility and permeability in drug discovery and development settings//Adv. Drug Deliv. Rev. Elsevier BV, 1997. Vol. 23, No 1-3. P. 3-25.
14. Bonnet P. Is chemical synthetic accessibility computationally predictable for drug and lead-like molecules?A comparative assessment between medicinal and computational chemists//Eur. J. Med. Chem. Elsevier, 2012. Vol. 54. P. 679-689.
15. Uspensky J. V. Introduction to mathematical probability//(No Title). cir.nii.ac.jp, 1937.
16. Boda K., Johnson A. P. Molecular complexity analysis of de novo designed ligands//J. Med. Chem. ACS Publications, 2006. Vol. 49, NX 20. P. 5869-5879.
17. Boda K., Seidel T., Gasteiger J. Structure and reaction based evaluation of synthetic accessibility//J. Comput. Aided Mol. Des. Springer, 2007. Vol. 21, No 6. P. 311-325.
18. Ertl P., Schuffenhauer A. Estimation of synthetic accessibility score of drug-like molecules based on molecular complexity and fragment contributions//J. Cheminform. Springer, 2009. Vol. 1, No 1. P. 8.
19. Vorsilik M. et al. SYBA: Bayesian estimation of synthetic accessibility of organic compounds//J. Cheminform. jcheminf.biomedcentral.com, 2020. Vol. 12, NX 1. P. 35.
20. Zagribelnyy B. et al. Retrosynthesis-related synthetic accessibility: pat. 2021229454:A1 USA//World Patent. 2021.
21. Ivanenkov Y. A. et al. Chemistry42: An AI-Driven Platform for Molecular Design and Optimization//J. Chem. Inf. Model. ACS Publications, 2023. Vol. 63, No 3. P. 695-701.
22. Jensen J. H. A graph-based genetic algorithm and generative model/Monte Carlo tree search for the exploration of chemical space//Chem. Sci. pubs.rsc.org, 2019. Vol. 10, NX 12. P. 3567-3572.
23. Hernindez-Lobato J. M., Sanchez-Lengeling B. Automatic chemical design using a data-driven continuous representation of molecules//ACS central. ACS Publications, 2018.
24. Polykovskiy D. et al. Molecular Sets (MOSES): A Benchmarking Platform for Molecular Generation Models//Front. Pharmacol. frontiersin.org, 2020. Vol. 11. P. 565644.
25. Coley C. W. et al. SCScore: Synthetic Complexity Learned from a Reaction Corpus//J. Chem. Inf. Model. ACS Publications, 2018. Vol. 58, No 2. P. 252-261.
26. Ertl P. Magic Rings: Navigation in the Ring Chemical Space Guided by the Bioactive Rings//J. Chem. Inf. Model. 2022. Vol. 62, NX 9. P. 2164-2170.
27. Kabir E., Uzzaman M. A review on biological and medicinal impact of heterocyclic compounds//Results Chem. Elsevier BV, 2022. Vol. 4, NX 100606. P. 100606.
28. Segler M. H. S. et al. Generating Focused Molecule Libraries for Drug Discovery with Recurrent Neural Networks//ACS Cent Sci. ACS Publications, 2018. Vol. 4, No 1. P. 120-131.
29. Skalic M. et al. Shape-Based Generative Modeling for de Novo Drug Design//J. Chem. Inf. Model. ACS Publications, 2019. Vol. 59, NX 3. P. 1205-1214.
30. Srinivasan S. et al. Artificial Intelligence-Guided De Novo Molecular Design Targeting COVID-19//ACS Omega. ACS Publications, 2021. Vol. 6, No 19. P. 12557-12566.
31. Zhavoronkov A. et al. Deep learning enables rapid identification of potent DDR1 kinase inhibitors//Nat. Biotechnol. nature.com, 2019. Vol. 37, No 9. P. 1038-1040.
32. Creanza T. M. et al. DeLA-Drug: A Deep Learning Algorithm for Automated Design of Druglike Analogues//J. Chem. Inf. Model. ACS Publications, 2022. Vol. 62, No 6. P. 1411-1424.
33. Krishnan S. R. et al. De novo design of anti-tuberculosis agents using a structure-based deep learning method//J. Mol. Graph. Model. Elsevier, 2023. Vol. 118. P. 108361.
34. Krishnan S. R. et al. De Novo Structure-Based Drug Design Using Deep Learning//J. Chem. Inf Model. ACS Publications, 2022. Vol. 62. No 21. P. 5100-5109.
35. Bung N., Krishnan S. R., Roy A. An In Silico Explainable Multiparameter Optimization Approach for De Novo Drug Design against Proteins from the Central Nervous System//J. Chem. Inf. Model. ACS Publications, 2022. Vol. 62, NX 11. P. 2685-2695.
36. Krishnan S. R. et al. Accelerating De Novo Drug Design against Novel Proteins Using Deep Learning//J. Chem. Inf. Model. ACS Publications, 2021. Vol. 61, No 2. P. 621-630.
37. ChEMBL Database. URL: www.ebi.ac.uk/chembl/
38. ZINC. URL: zinc.docking.org/
39. PubChem. PubChem. URL: pubchem.ncbi.nlm.nih.gov/
40. Screening collections—Enamine. URL: enamine.net/compound-collections/screening-collection
41. WuXi Biologics. URL: www.wuxibiologics.com/
42. SciFinder®. URL: scifinder-n.cas.org/
43. Reaxys. URL: elsevier.com/solutions/reaxys
44. Baell J. B., Nissink J. W. M. Seven year itch: Pan-assay interference compounds (PAINS) in 2017-utility and limitations//ACS Chem. Biol. American Chemical Society (ACS), 2018. Vol. 13, NX 1. P. 36-44.
45. Ertl P. Database of 4 Million Medicinal Chemistry-Relevant Ring Systems//J. Chem. Inf. Model. 2024.
46. Vitaku E., Smith D. T., Njardarson J. T. Analysis of the structural diversity, substitution patterns, and frequency of nitrogen heterocycles among U.S. FDA approved pharmaceuticals//J. Med. Chem. American Chemical Society (ACS), 2014. Vol. 57, No 24. P. 10257-10274.
47. SMARTSTM—A Language for Describing Molecular Patterns//Daylight Chemical Information Systems, Inc. 2007. URL: www.daylight.com/dayhtml/doc/theory/theory.smarts.html.
48. Weininger D. SMILES, a chemical language and information system. 1. Introduction to methodology and encoding rules//J. Chem. Inf. Comput. Sci. American Chemical Society, 1988. Vol. 28, No 1. P. 31-36.
49. Rdkit: Open-Source Cheminformatics Tool. URL: www.Rdkit.org
50. Mendez D. et al. ChEMBL: towards direct deposition of bioassay data//Nucleic Acids Res. Oxford University Press (OUP), 2019. Vol. 47, No D1. P. D930-D940.
51. Chemistry42. URL: insilico.com/chemistry42
52. Wei L. et al. Cov_FB3D: A De Novo Covalent Drug Design Protocol Integrating the BA-SAMP Strategy and Machine-Learning-Based Synthetic Tractability Evaluation//J. Chem. Inf. Model. ACS Publications, 2020. Vol. 60, No 9. P. 4388-4402.
53. Bung N. et al. De novo design of new chemical entities for SARS-CoV-2 using artificial intelligence//Future Med. Chem. Future Science, 2021. Vol. 13, NX 6. P. 575-585.
54. Srinivas R. et al. Deep Learning-Based Ligand Design Using Shared Latent Implicit Fingerprints from Collaborative Filtering//J. Chem. Inf. Model. ACS Publications, 2021. Vol. 61, No 5. P. 2159-2174.
55. Bilsland A. E. et al. Automated Generation of Novel Fragments Using Screening Data, a Dual SMILES Autoencoder, Transfer Learning and Syntax Correction//J. Chem. Inf. Model. ACS Publications, 2021. Vol. 61, No 6. P. 2547-2559.
56. Yang L. et al. Transformer-Based Generative Model Accelerating the Development of Novel BRAF Inhibitors//ACS Omega. ACS Publications, 2021. Vol. 6, No 49. P. 33864-33873.
57. Hu L. et al. Kinase Inhibitor Scaffold Hopping with Deep Learning Approaches//J. Chem. Inf. Model. ACS Publications, 2021. Vol. 61, No 10. P. 4900-4912.
58. Zabolotna Y. et al. SynthI: A New Open-Source Tool for Synthon-Based Library Design//J. Chem. Inf Model. ACS Publications, 2022. Vol. 62. No 9. P. 2151-2163.

1. A method for improving a synthetic accessibility analysis of molecular structures, comprising:
generating a hierarchical library of molecule substructures representing possible molecular fragments;
collecting statistics on substructure occurrences in reference chemical datasets;
identifying synthetically irrelevant substructures based on the occurrence statistics, wherein low occurrence statistics indicate the synthetically irrelevant substructures;
clustering and optimizing the library of irrelevant substructures;
filtering a group of molecular structures using a hierarchical algorithm to identify those containing irrelevant substructures; and
removing the molecular structures containing irrelevant substructures from the group of molecular structures to obtain a filtered set of molecular structures that are filtered by removing molecular structures having synthetically irrelevant substructures.

2. The method of claim 1, wherein generating the hierarchical library comprises:
defining a zero layer comprising a defined genus of a chemical moiety, optionally as a basic aromatic five-membered ring;
generating a first layer by combinatorically replacing atoms in the zero layer with atomic primitives from an element alphabet;
generating a second layer by replacing atoms with atomic primitives of a different type;
generating a third layer by replacing a defined symbol with a hydrogen atom in the second-layer strings; and
generating at least one subsequent layer by replacing substituents according to a substituent alphabet.

3. The method of claim 1, wherein collecting statistics comprises:
analyzing substructure occurrences in reference datasets of synthesized compounds; and
determining frequency thresholds for classifying substructures as synthetically relevant or irrelevant.

4. The method of claim 1, wherein clustering and optimizing the library comprises:
combining homogeneous substituents in SMARTS strings to reduce the number of substructures.

5. The method of claim 1, wherein the hierarchical filtering algorithm comprises:
checking for the presence of the defined genus of a chemical moiety, optionally a five-membered aromatic heterocycle;
if present, checking for substructures from the first layer of the library;
proceeding to subsequent layers if matches are found; and
terminating and rejecting the structure if a match is found in the final layer.

6. The method of claim 1, further comprising:
receiving a molecular structure represented as a SMILES string;
parsing the SMILES string to generate a molecular graph object;
applying a hierarchical filtering algorithm to the molecular graph object, wherein the hierarchical filtering algorithm comprises:
querying the molecular graph object against a zero layer of a hierarchical library to determine presence of a target core structure;
when the target core structure is present, sequentially querying the molecular graph object against successive layers of the hierarchical library, wherein each successive layer represents progressively more specific structural features of the target core structure with local chemical environment;
terminating the hierarchical filtering algorithm upon detecting a match with a synthetically irrelevant substructure from a final layer of the hierarchical library; and
generating a filtering result indicating whether the molecular structure contains a synthetically irrelevant substructure based on the hierarchical filtering algorithm.

7. The method of claim 1, wherein the hierarchical filtering algorithm terminates without applying a penalty when:
the target core structure is absent from the molecular graph object; or
no match is found in any layer of the hierarchical library.

8. The method of claim 1, further comprising:
when a synthetically irrelevant substructure is detected, retrieving atom mapping indices corresponding to the synthetically irrelevant substructure within the molecular graph object; and
generating a visualization highlighting the synthetically irrelevant substructure based on the atom mapping indices.

9. The method of claim 1, wherein the filtering result comprises a Boolean value indicating PASS when no synthetically irrelevant substructure is detected and FILTER when a synthetically irrelevant substructure is detected.

10. A computer-implemented method for constructing a hierarchical library of molecular substructures for synthetic accessibility evaluation, the method comprising:
defining a zero layer comprising a core structure represented as a SMARTS string;
defining an element alphabet comprising atomic primitives for heteroatoms;
generating a first layer by combinatorially replacing generic atom symbols in the zero layer SMARTS string with atomic primitives from the element alphabet to produce heterocycle type substructures;
defining a substituent alphabet comprising bond primitives and atomic primitives for substituent types;
generating a second layer by replacing attachment point symbols in the first layer substructures with hydrogen atoms and substituent placeholders to produce position-specific substructures;
generating a third layer by enumerating substructures having two or three substituent positions;
generating a fourth layer by combinatorially substituting all substituent types from the substituent alphabet at each substituent position in the third layer substructures;
collecting occurrence statistics for each substructure in the fourth layer by querying a reference dataset of synthesized compounds;
identifying synthetically irrelevant substructures as those having occurrence frequency below a predetermined threshold; and
storing the hierarchical library comprising the zero layer, first layer, second layer, third layer, and fourth layer with associated occurrence statistics.

11. The method of claim 10, further comprising:
removing duplicate substructures by performing isomorphism checks between molecular graph representations of the substructures.

12. The method of claim 10, further comprising:
clustering homogeneous substituent variations in the fourth layer by:
identifying groups of substructures differing only in substituent type at one or more positions;
combining atomic primitives for the varying substituents using logical OR operators; and
replacing the groups of substructures with single clustered SMARTS strings.

13. A computer-implemented method for filtering molecular structures generated by a generative chemistry model, the method comprising:
receiving a batch of molecular structures from a generative chemistry model, each molecular structure represented as a SMILES string;
for each molecular structure in the batch:
parsing the SMILES string to generate a molecular graph object;
applying a hierarchical filtering algorithm to determine whether the molecular graph object contains a synthetically irrelevant substructure by traversing a hierarchical library of SMARTS-encoded substructures;
assigning a PASS status when no synthetically irrelevant substructure is detected; and
assigning a FILTER status when a synthetically irrelevant substructure is detected;
generating a filtered set comprising molecular structures having PASS status; and
outputting the filtered set for synthesis prioritization.

14. The method of claim 13, further comprising:
for each molecular structure having FILTER status, identifying the synthetically irrelevant substructure and corresponding atom mapping indices; and
generating a report comprising statistics on violation types and frequencies.

15. The method of claim 13, further comprising:
providing feedback to the generative chemistry model comprising:
a list of synthetically irrelevant substructures to avoid; and
occurrence statistics from a reference dataset for each synthetically irrelevant substructure.

16. The method of claim 13, further comprising:
obtaining a target molecule having a molecular structure devoid of synthetically irrelevant substructures; and
synthesizing the target molecule according to a synthetic route.

17. The method of claim 16, further comprising:
validating the synthesized target molecule by at least one of:
chemical validation comprising spectroscopic analysis; or
biological validation comprising activity assay.

18. A computer-implemented method for selecting a synthetic route for a target molecule, the method comprising:
receiving a target molecule represented as a SMILES string;
generating a plurality of candidate synthetic routes for the target molecule;
for each candidate synthetic route:
identifying intermediate molecular structures;
applying a hierarchical filtering algorithm to each intermediate molecular structure to determine presence of synthetically irrelevant substructures;
calculating a synthetic accessibility score based on number of intermediate molecular structures containing synthetically irrelevant substructures;
selecting a preferred synthetic route having a lowest synthetic accessibility score; and
outputting the preferred synthetic route for synthesis execution.

19. The method of claim 18, further comprising:
synthesizing the target molecule according to the preferred synthetic route.

20. The method of claim 19, further comprising:
validating the synthesized target molecule by at least one of:
chemical validation comprising spectroscopic analysis; or
biological validation comprising activity assay.

21. A system for improving a synthetic accessibility analysis of molecular structures, comprising:
one or more processors; and
memory storing instructions that, when executed by the one or more processors, cause the system to perform the method of claim 1.

22. A system for improving synthetic accessibility of molecular structures, comprising:
one or more processors; and
memory storing instructions that, when executed by the one or more processors, cause the system to perform the method of claim 11.

23. A non-transitory computer-readable medium storing instructions that, when executed by one or more processors, cause performance of the method of claim 1.

24. A non-transitory computer-readable medium storing instructions that, when executed by one or more processors, cause performance of the method of claim 11.
